SEQUENCE LISTING (6 sequences)

SEQ ID NO: 1 - protein, 595 aa, Mus musculus
LEVPTDGNAGLLAEPQIAMFCGKLNMHMNVQNGKWESDPSGTKTCIGTKEGILQYCQEVYPELQITNVVEANQPVTIQNWCKRGRKQCKTHTHIVIPYRCLVGEFVSDALLVPDKCKFLHQERMDVCETHLHWHTVAKETCSEKSTNLHDYGMLLPCGIDKFRGVEFVCCPLAEESDSVDSADAEEDDSDVWWGGADTDYADGGEDKVVEVAEEEEVADVEEEEADDDEDVEDGDEVEEEAEEPYEEATERTTSTATTTTTTTESVEEVVRVPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPHHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHPFGVDSVPANTENEVEPVDARPAADRGLTTRPGSGLTNIKTEEISEVKMDAEFGHDSGFEVRHQK

SEQ ID NO: 2 - protein, 595 aa, Homo sapiens
LEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVRVPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQK

SEQ ID NO: 3 - DNA, 1785 bp, Artificial Sequence
  Synthetic codon-optimized nucleic acid sequence
ttggaggtgcccaccgacggcaacgctggactgctggctgaaccccagatcgccatgttctgcggcaagctgaacatgcacatgaacgtgcagaacggcaagtgggagagcgaccccagcggcaccaagacctgcatcggcaccaaagagggcatcctgcagtattgccaggaagtgtaccccgagctgcagatcaccaacgtggtggaagccaaccagcccgtgaccatccagaactggtgcaagaggggcagaaagcagtgcaagacccacacccacatcgtgatcccttacagatgcctcgtgggcgagttcgtgtccgacgctctgctggtgcccgacaagtgcaagttcctgcatcaggaacggatggacgtgtgcgagacacatctgcactggcacaccgtggccaaagagacatgcagcgagaagtccaccaacctgcacgactacggcatgctgctgccctgcggcatcgacaagttcagaggcgtggaattcgtgtgctgccccctggccgaggaatccgactctgtggatagcgccgacgccgaagaggacgactctgacgtgtggtggggcggagccgacacagattacgctgatggcggcgaggacaaggtggtggaagtggctgaagaggaagaggtggccgacgtggaagaagaagaggccgacgacgacgaggatgtggaagatggcgacgaggtggaagaggaagccgaggaaccctacgaggaagccaccgagagaaccaccagcaccgccaccacaaccaccaccactaccgagagcgtggaagaggtcgtgcgggtgccaacaacagccgcctctacacctgacgccgtggacaagtacctggaaaccccaggcgacgagaacgagcacgcccacttccagaaggctaaagagagactggaagctaagcaccgcgagagaatgagccaagtgatgagagagtgggaggaagctgagagacaggccaagaacctgcccaaggccgacaagaaagccgtgatccagcacttccaggaaaaggtggaaagcctggaacaggaagctgccaacgagagacagcagctggtggaaacccacatggccagagtggaagctatgctgaacgacagaagaaggctggccctggaaaactacatcaccgctctgcaggccgtgccccccagacctcaccacgtgttcaacatgctgaagaaatacgtgcgggccgagcagaaggacagacagcacaccctgaagcacttcgagcacgtgcggatggtggaccccaagaaggccgcccagatcagatcccaagtgatgacccacctgagagtgatctacgagaggatgaaccagagcctgagcctgctgtacaacgtgcccgccgtggccgaagaaatccaggatgaggtggacgagctgctgcagaaagagcagaactacagcgacgacgtgctggccaacatgatcagcgagcccagaatcagctacggcaacgacgccctgatgcccagcctgaccgagacaaagaccaccgtggaactgctgcccgtgaacggcgagttcagcctggatgacctgcagccctggcaccctttcggcgtggactctgtgcctgccaacacagagaacgaagtggaacccgtggacgccagacctgccgctgatagaggcctgaccacaagacctggcagcggcctgacaaacatcaagaccgaagagatcagcgaagtgaagatggacgccgagttcgggcacgacagcggctttgaagtgcggcaccagaaa

SEQ ID NO: 4 - DNA, 1785 bp, Artificial Sequence
  Synthetic codon-optimized nucleic acid sequence
ttggaggtgcccaccgacggcaacgccggactgctggccgagccccagatcgccatgttctgcggcagactgaacatgcacatgaacgtgcagaacggcaagtgggacagcgaccccagcggcaccaagacctgcatcgacaccaaagagggcatcctgcagtattgccaagaagtgtaccccgagctgcagatcaccaacgtggtggaagccaaccagcccgtgaccatccagaactggtgcaagcggggcagaaagcagtgcaagacccacccccacttcgtgatcccttacagatgcctcgtgggcgagttcgtgtccgacgccctgctggtgcccgacaagtgcaagttcctgcatcaagaacggatggacgtgtgcgagacacatctgcactggcacaccgtggccaaagagacatgcagcgagaagtccaccaacctgcacgactacggcatgctgctgccctgcggcatcgacaagttccggggcgtggaattcgtgtgctgccccctggccgaggaatccgacaacgtggacagcgccgacgccgaagaggacgacagcgacgtgtggtggggcggagccgacaccgattacgccgacggcagcgaggacaaggtggtggaagtggctgaagaggaagaggtggccgaggtcgaggaagaggaagccgacgacgacgaggatgacgaggacggcgacgaggtggaagaagaggccgaggaaccctacgaggaagccaccgagcggaccacctctatcgccaccaccaccacaaccactaccgagagcgtggaagaggtcgtgcgggtgccaaccaccgccgcctctacccccgacgccgtggacaagtacctggaaacccctggcgacgagaacgagcacgcccacttccagaaggccaaagagcggctggaagccaagcaccgcgagcggatgagccaggtcatgagagagtgggaagaagccgagcggcaggccaagaacctgcccaaggccgacaagaaagccgtgatccagcacttccaagaaaaggtcgagagcctggaacaagaagccgccaacgagcggcagcagctggtggaaacccacatggccagagtggaagccatgctgaacgaccggcggagactggccctggaaaactacatcaccgctctgcaggccgtgccccccagaccccggcacgtgttcaacatgctgaagaaatacgtgcgggccgagcagaaggaccggcagcacaccctgaagcacttcgagcacgtgcggatggtggaccccaagaaggccgcccagatccgctctcaggtcatgacccacctgagagtgatctacgagagaatgaaccagagcctgagcctgctgtacaacgtgcccgccgtggccgaagaaatccaggatgaggtggacgagctgctgcagaaagagcagaactacagcgacgacgtgctggccaacatgatcagcgagccccggatcagctacggcaacgacgccctgatgcccagcctgaccgagacaaagaccaccgtggaactgctgcccgtgaacggcgagttcagcctggacgacctgcagccctggcacagcttcggcgctgatagcgtgcccgccaacaccgagaatgaggtggaacccgtggacgccagacctgccgccgatagaggcctgaccacaagacctggcagcggcctgaccaacatcaagaccgaagagatcagcgaagtgaagatggacgccgagttccggcacgacagcggctacgaggtgcaccaccagaaa

SEQ ID NO: 5 - DNA, 3740 bp, Artificial Sequence
  Synthetic complete sequence of the expression cassette
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctaggatcacgcgttctagaaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgaattgctagcggggatccaccggtcgccaccatgctgccttctctggctttgctgctgctggccgcttggacagtgcgggcctacccttacgacgtgcccgactacgcttacccctacgatgtgcctgattatgcattggaggtgcccaccgacggcaacgctggactgctggctgaaccccagatcgccatgttctgcggcaagctgaacatgcacatgaacgtgcagaacggcaagtgggagagcgaccccagcggcaccaagacctgcatcggcaccaaagagggcatcctgcagtattgccaggaagtgtaccccgagctgcagatcaccaacgtggtggaagccaaccagcccgtgaccatccagaactggtgcaagaggggcagaaagcagtgcaagacccacacccacatcgtgatcccttacagatgcctcgtgggcgagttcgtgtccgacgctctgctggtgcccgacaagtgcaagttcctgcatcaggaacggatggacgtgtgcgagacacatctgcactggcacaccgtggccaaagagacatgcagcgagaagtccaccaacctgcacgactacggcatgctgctgccctgcggcatcgacaagttcagaggcgtggaattcgtgtgctgccccctggccgaggaatccgactctgtggatagcgccgacgccgaagaggacgactctgacgtgtggtggggcggagccgacacagattacgctgatggcggcgaggacaaggtggtggaagtggctgaagaggaagaggtggccgacgtggaagaagaagaggccgacgacgacgaggatgtggaagatggcgacgaggtggaagaggaagccgaggaaccctacgaggaagccaccgagagaaccaccagcaccgccaccacaaccaccaccactaccgagagcgtggaagaggtcgtgcgggtgccaacaacagccgcctctacacctgacgccgtggacaagtacctggaaaccccaggcgacgagaacgagcacgcccacttccagaaggctaaagagagactggaagctaagcaccgcgagagaatgagccaagtgatgagagagtgggaggaagctgagagacaggccaagaacctgcccaaggccgacaagaaagccgtgatccagcacttccaggaaaaggtggaaagcctggaacaggaagctgccaacgagagacagcagctggtggaaacccacatggccagagtggaagctatgctgaacgacagaagaaggctggccctggaaaactacatcaccgctctgcaggccgtgccccccagacctcaccacgtgttcaacatgctgaagaaatacgtgcgggccgagcagaaggacagacagcacaccctgaagcacttcgagcacgtgcggatggtggaccccaagaaggccgcccagatcagatcccaagtgatgacccacctgagagtgatctacgagaggatgaaccagagcctgagcctgctgtacaacgtgcccgccgtggccgaagaaatccaggatgaggtggacgagctgctgcagaaagagcagaactacagcgacgacgtgctggccaacatgatcagcgagcccagaatcagctacggcaacgacgccctgatgcccagcctgaccgagacaaagaccaccgtggaactgctgcccgtgaacggcgagttcagcctggatgacctgcagccctggcaccctttcggcgtggactctgtgcctgccaacacagagaacgaagtggaacccgtggacgccagacctgccgctgatagaggcctgaccacaagacctggcagcggcctgacaaacatcaagaccgaagagatcagcgaagtgaagatggacgccgagttcgggcacgacagcggctttgaagtgcggcaccagaaatagaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaaccccccccccccccccc

SEQ ID NO: 6 - DNA, 3740 bp, Artificial Sequence
  Synthetic complete sequence of the expression cassette
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctaggatcacgcgttctagaaatattaaggtacgggaggtacttggagcggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgaattgctagcggggatccaccggtcgccaccatgctgcctggactggctctgctgctgctggccgcctggacagccagagcctacccctacgacgtgcccgactacgcctacccttatgatgtgcctgactatgcattggaggtgcccaccgacggcaacgccggactgctggccgagccccagatcgccatgttctgcggcagactgaacatgcacatgaacgtgcagaacggcaagtgggacagcgaccccagcggcaccaagacctgcatcgacaccaaagagggcatcctgcagtattgccaagaagtgtaccccgagctgcagatcaccaacgtggtggaagccaaccagcccgtgaccatccagaactggtgcaagcggggcagaaagcagtgcaagacccacccccacttcgtgatcccttacagatgcctcgtgggcgagttcgtgtccgacgccctgctggtgcccgacaagtgcaagttcctgcatcaagaacggatggacgtgtgcgagacacatctgcactggcacaccgtggccaaagagacatgcagcgagaagtccaccaacctgcacgactacggcatgctgctgccctgcggcatcgacaagttccggggcgtggaattcgtgtgctgccccctggccgaggaatccgacaacgtggacagcgccgacgccgaagaggacgacagcgacgtgtggtggggcggagccgacaccgattacgccgacggcagcgaggacaaggtggtggaagtggctgaagaggaagaggtggccgaggtcgaggaagaggaagccgacgacgacgaggatgacgaggacggcgacgaggtggaagaagaggccgaggaaccctacgaggaagccaccgagcggaccacctctatcgccaccaccaccacaaccactaccgagagcgtggaagaggtcgtgcgggtgccaaccaccgccgcctctacccccgacgccgtggacaagtacctggaaacccctggcgacgagaacgagcacgcccacttccagaaggccaaagagcggctggaagccaagcaccgcgagcggatgagccaggtcatgagagagtgggaagaagccgagcggcaggccaagaacctgcccaaggccgacaagaaagccgtgatccagcacttccaagaaaaggtcgagagcctggaacaagaagccgccaacgagcggcagcagctggtggaaacccacatggccagagtggaagccatgctgaacgaccggcggagactggccctggaaaactacatcaccgctctgcaggccgtgccccccagaccccggcacgtgttcaacatgctgaagaaatacgtgcgggccgagcagaaggaccggcagcacaccctgaagcacttcgagcacgtgcggatggtggaccccaagaaggccgcccagatccgctctcaggtcatgacccacctgagagtgatctacgagagaatgaaccagagcctgagcctgctgtacaacgtgcccgccgtggccgaagaaatccaggatgaggtggacgagctgctgcagaaagagcagaactacagcgacgacgtgctggccaacatgatcagcgagccccggatcagctacggcaacgacgccctgatgcccagcctgaccgagacaaagaccaccgtggaactgctgcccgtgaacggcgagttcagcctggacgacctgcagccctggcacagcttcggcgctgatagcgtgcccgccaacaccgagaatgaggtggaacccgtggacgccagacctgccgccgatagaggcctgaccacaagacctggcagcggcctgaccaacatcaagaccgaagagatcagcgaagtgaagatggacgccgagttccggcacgacagcggctacgaggtgcaccaccagaaatagaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaaccccccccccccccccc

(19) United States
(12) Patent Application Publication
(10) Pub. No.: US 2018/0161395 A1
(43) Pub. Date: Jun. 14, 2018

(54) METHODS AND PHARMACEUTICAL COMPOSITION FOR THE TREATMENT OF ALZHEIMER'S DISEASE

(71) Applicants: INSERM (INSTITUT NATIONAL DE LA SANTE ET DE LA RECHERCHE MEDICALE), Paris (FR); UNIVERSITE PARIS-SUD, Orsay (FR); COMMISSARIAT A L'ENERGIE ATOMIQUE ET AUX ENERGIES ALTERNATIVES, Paris (FR); UNIVERSITE PARIS DESCARTES, Paris (FR); UNIVERSITAT HEIDELBERG, Heidelberg (DE)

(72) Inventors: Romain FOL, Fontenay Aux Roses Cedex (FR); Jerome BRAUDEAU, Fontenay Aux Roses (FR); Nathalie CARTIER, Fontenay Aux Roses (FR); Christian BUCHHOLZ, Langen (DE); Abel TOBIAS, Langen (DE); Ulrike MUELLER, Heidelberg (DE)

(21) Appl. No.: 15/580,934

(22) PCT Filed: Jun. 10, 2016
(86) PCT No.: PCT/EP2016/063338

§ 371 Date: Dec. 8, 2017

(30) Foreign Application Priority Data
Jun. 12, 2015 (EP) 15305904.3

(51) Int. Cl. A61K 38/17; A61K 48/00; A61K 35/28; A61P 25/28

(52) U.S. Cl. CPC A61K 38/1716; A61K 48/005; A61P 25/28; A61K 35/28; A61K 48/0075

(57) ABSTRACT

The present invention relates to methods and pharmaceutical compositions for the treatment of Alzheimer's disease. In particular the present invention relates to a method of treating Alzheimer's disease in a subject in need thereof comprising administering to the subject a therapeutically effective amount of a vector which comprises a nucleic acid molecule encoding for a polypeptide which is a soluble member of the APP (amyloid precursor protein) family.

### FIELD OF THE INVENTION

The present invention relates to methods and pharmaceutical compositions for the treatment of Alzheimer's disease.

### BACKGROUND OF THE INVENTION

Synaptic dysfunction, cognitive decline, and excessive accumulation of neurotoxic β-amyloid peptides (Aβ), are hallmark features of Alzheimer's disease (AD). Aβ is generated by sequential cleavage of the amyloid precursor protein (APP) by β- and γ-secretase. In the competing and physiologically predominant non-amyloidogenic pathway α-secretase cleaves APP within the Aβ region (Lichtenthaler et al, 2011; Prox et al, 2012) thus precluding the formation of Aβ peptides. This leads to the secretion of the neuroprotective ectodomain APPsα, into the extracellular space in a process that can be stimulated by neuronal and synaptic activity (Hoe et al, 2012; Hoey et al, 2009).

Processing of APP by β-secretase within the amyloidogenic pathway leads to the generation of the large ectodomain APPsβ and membrane bound stubs termed CTFβ. CTFβ is then further cleaved by γ-secretase leading to the production of Aβ. AD is characterized by upregulation of β-secretase (BACE-1) resulting in a shift towards amyloidogenic APP processing (Ahmed et al, 2010; Holsinger et al, 2002). Increasing evidence suggests that the concomitant reduction in APPsα and the loss of its physiological functions contributes to AD pathogenesis. Reduced levels of APPsα or ADAM10 were reported in patients with amyloid deposits and AD (Dobrowolska et al, 2014; Lannfelt et al, 1995) and reviewed in (Endres & Fahrenholz, 2012). Lowered levels of CSF APPsα were also correlated with poor memory performance in both human patients and aged rats (reviewed in Endres & Fahrenholz, 2012). Moreover, α-secretase attenuating mutations have been associated with hereditary late-onset AD (Suh et al, 2013). Interestingly, APPsα has recently also been shown to reduce Aβ generation by binding to and thereby inhibiting BACE-1 activity (Obregon et al, 2012).

Substantial evidence has implicated APP and APPsα in protecting cultured neurons in vitro against various forms of stress (Kogel et al, 2012). In vivo, APP was found upregulated in response to brain injury suggesting a role in damage response (Leyssen et al, 2005; Murakami et al, 1998; Ramirez et al, 2001; Van den Heuvel et al, 1999). Importantly, in addition to neuroprotection APPsα has prominent physiological functions for neurite outgrowth, synaptogenesis, adult neurogenesis, synaptic plasticity and hippocampus-dependent behavior (Aydin et al, 2012). Previously, the inventors generated APP-KO mice that showed impaired long-term potentiation (LTP) and spatial learning that was fully rescued in APPsα knock-in mice expressing solely APPsα from the endogenous APP locus (Ring et al, 2007). Recently, to avoid functional redundancy within the APP gene family (reviewed in (Aydin et al, 2012), we generated conditional double knockout mice (cDKO) lacking APP and the close homologue APLP2 in excitatory forebrain neurons (Hick et al, 2015). These cDKO mice revealed reduced spine density and impaired synaptic plasticity that was associated with deficits in hippocampus dependent behaviors (Hick et al 2015).

Some studies showed the possible implication of APPsα in neuroprotection and neuromodulation but were limited to specific situations (acute brain injury for example) and no prove of concept on Alzheimer was demonstrated (Corrigan et al, 2012; Thornton et al, 2006).

Some other strategies to treat AD targeting α-secretase ADAM-10 were tested but with poor specificity (ADAM-10 has several hundred other substrates) and efficacy (Kuhn et al., 2015).

Thus; there is still a need for an effective and safe AD treatment strategy.

### SUMMARY OF THE INVENTION

The present invention relates to methods and pharmaceutical compositions for the treatment of Alzheimer's disease. In particular, the present invention is defined by the claims.

### DETAILED DESCRIPTION OF THE INVENTION

Here, the inventors used direct overexpression of APPsα by AAV-mediated gene transfer into the brain to explore its potential to ameliorate or rescue structural, electrophysiological and behavioral deficits of AD model mice. Unexpectedly, they found that overexpression of APPsα in aged transgenic APP/PS1ΔE9 mice with well-established plaque pathology improves synaptic plasticity and partially rescues spine density deficits. Restoration of synaptic plasticity and increased spine density is also accompanied by a rescue of spatial memory. Moreover, APPsα expression leads to moderately reduced Aβ levels and significantly ameliorated plaque pathology. Interestingly, in AAV-APPsα injected mice, they observed an increased recruitment of microglia towards plaques which may have led to increased plaque clearance. Collectively, these data suggest, that even at stages with advanced plaque deposition APPsα may counteract Aβ induced synaptotoxic effects and restores cognitive functions. Unexpectedly, in contrast to AAV-APPsα, the inventors show that AAV-APPβ injection is not able to restore memory deficits of APP/PS1ΔE9 and that APPsβ expression in contrast to APPsα expression fails to ameliorate functional synaptic impairments of aged AD model mice. Also, in contrast to APPsα, APPsβ fails to increase microglia density in the vicinity to plaques and induced no upregulation of the microglia marker Iba1.

Accordingly, a first object of the present invention relates to a method of treating Alzheimer's disease in a subject in need thereof comprising administering to the subject a therapeutically effective amount of a vector which comprises a nucleic acid molecule encoding for a polypeptide which is a soluble member of the APP (amyloid precursor protein) family.

Another object of the present invention relates to a method of treating Down syndrome in a subject in need thereof comprising administering to the subject a therapeutically effective amount of a vector which comprises a nucleic acid molecule encoding for a polypeptide which is a soluble member of the APP (amyloid precursor protein) family.

As used herein, the term “subject” denotes a mammal, such as a rodent, a feline, a canine, and a primate. Preferably a subject according to the invention is a human. In the context of the present invention, a “subject in need thereof” denotes a subject, preferably a human, with Alzheimer's disease, prodromal Alzheimer's or Down syndrome (Trisomy 21).

As used herein, the term “Alzheimer's disease” has its general meaning in the art and denotes chronic neurodegenerative disease that usually starts slowly and gets worse over time. Alzheimer's disease (AD) is characterized by amyloid deposits, intracellular neurofibrillary tangles, neuronal loss and a decline in cognitive function. The most common early symptom is difficulty in remembering recent events (short-term memory loss). As the disease advances, symptoms can include: problems with language, disorientation (including easily getting lost), mood swings, loss of motivation, not managing self-care, and behavioural issues. AD is undoubtedly multifactorial, but the amyloid protein precursor (APP) is a key element in its development. The physiological functions of APP of its first cleavage product APPsα are unclear, but it has been shown to play crucial roles for spine density, morphology and plasticity. As used herein, the term “prodromal Alzheimer's” refers to the very early form of Alzheimer's when memory is deteriorating but a person remains functionally independent.

As used herein, the term “Down syndrome” has its general meaning in the art and refers to a genetic disorder caused by the presence of all, or part of a third copy of chromosome 21. It is typically associated with physical growth delays, characteristic facial features, and mild to moderate intellectual disability. The Down syndrome is also called trisomy 21.

As used herein, the term “treatment” or “treat” refers to both prophylactic or preventive treatment as well as curative or disease modifying treatment, including treatment of subjects at risk of contracting the disease or suspected to have contracted the disease as well as subjects who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

In particular, the method of the present invention is particularly suitable for rescuing memory impairment, synaptic plasticity and/or spine density, ameliorating both structural and functional synaptic impairments, decreasing Aβ levels and plaque deposition, inducing microglia recruitment and activation in the vicinity of amyloid plaques, enhancing Aβ and plaque clearance and/or restoring cognitive functions.

As used herein the “amyloid precursor protein (APP) family” has its general meaning in the art and represents integral membrane proteins expressed in many tissues and concentrated in the synapses of neurons. Amyloid precursor proteins include APP, APLP1 (amyloid beta (A4) precursor-like protein 1) and APLP2 (amyloid beta (A4) precursor-like protein 1). Soluble members of the amyloid precursor protein (APP) family include the form cleaved by secretases. The soluble members thus include APPsα, APLP1s and APLP2s.

In some embodiments, the vector of the present invention comprises a nucleic acid encoding for an APPsα polypeptide.

As used herein the term “APPsα” has its general meaning in the art and refers to the protein formed by the cleavage of the amyloid precursor protein (APP) by the α-secretase. The APPsα is then secreted into the extracellular space. Exemplary amino acid sequences of APPsα include sequences a set forth in SEQ ID NO:1 and SEQ ID NO:2.

SEQ ID NO 1: amino acid sequence of the murine
APPsα protein
LEVPTDGNAGLLAEPQIAMFCGKLNMHMNVQNGKWESDPSGTKTCIGTKE
GILQYCQEVYPELQITNVVEANQPVTIQNWCKRGRKQCKTHTHIVIPYRC
LVGEFVSDALLVPDKCKFLHQERMDVCETHLHWHTVAKETCSEKSTNLHD
YGMLLPCGIDKFRGVEFVCCPLAEESDSVDSADAEEDDSDVWWGGADTDY
ADGGEDKVVEVAEEEEVADVEEEEADDDEDVEDGDEVEEEAEEPYEEATE
RTTSTATTTTTTTESVEEVVRVPTTAASTPDAVDKYLETPGDENEHAHFQ
KAKERLEAKHRERMSQVMREWEEAERQAKNLPKADKKAVIQHFQEKVESL
EQEAANERQQLVETHMARVEAMLNDRRRLALENYITALQAVPPRPHHVFN
MLKKYVRAEQKDRQHTLKHFEHVRMVDPKKAAQIRSQVMTHLRVIYERMN
QSLSLLYNVPAVAEEIQDEVDELLQKEQNYSDDVLANMISEPRISYGNDA
LMPSLTETKTTVELLPVNGEFSLDDLQPWHPFGVDSVPANTENEVEPVDA
RPAADRGLTTRPGSGLTNIKTEEISEVKMDAEFGHDSGFEVRHQK
SEQ ID NO: 2: amino acid sequence of the human
APPsα protein
LEVPTDGNAGLLAEPQIAMFCGRLNMHMNVQNGKWDSDPSGTKTCIDTKE
GILQYCQEVYPELQITNVVEANQPVTIQNWCKRGRKQCKTHPHFVIPYRC
LVGEFVSDALLVPDKCKFLHQERMDVCETHLHWHTVAKETCSEKSTNLHD
YGMLLPCGIDKFRGVEFVCCPLAEESDNVDSADAEEDDSDVWWGGADTDY
ADGSEDKVVEVAEEEEVAEVEEEEADDDEDDEDGDEVEEEAEEPYEEATE
RTTSIATTTTTTTESVEEVVRVPTTAASTPDAVDKYLETPGDENEHAHFQ
KAKERLEAKHRERMSQVMREWEEAERQAKNLPKADKKAVIQHFQEKVESL
EQEAANERQQLVETHMARVEAMLNDRRRLALENYITALQAVPPRPRHVFN
MLKKYVRAEQKDRQHTLKHFEHVRMVDPKKAAQIRSQVMTHLRVIYERMN
QSLSLLYNVPAVAEEIQDEVDELLQKEQNYSDDVLANMISEPRISYGNDA
LMPSLTETKTTVELLPVNGEFSLDDLQPWHSFGADSVPANTENEVEPVDA
RPAADRGLTTRPGSGLTNIKTEEISEVKMDAEFRHDSGYEVHHQK

In some embodiments, the vector of the present invention comprises a nucleic acid molecule encoding for a APPsα polypeptide comprising an amino acid sequence having at least 90% of identity with the sequence as set forth in SEQ ID NO:1 or 2.

According to the invention a first amino acid sequence having at least 90% of identity with a second amino acid sequence means that the first sequence has 90; 91; 92; 93; 94; 95; 96; 97; 98; 99 or 100% of identity with the second amino acid sequence.

Sequence identity is frequently measured in terms of percentage identity (or similarity or homology); the higher the percentage, the more similar are the two sequences. Methods of alignment of sequences for comparison are well known in the art. Various programs and alignment algorithms are described in: Smith and Waterman, Adv. Appl. Math., 2:482, 1981; Needleman and Wunsch, J. Mol. Biol., 48:443, 1970; Pearson and Lipman, Proc. Natl. Acad. Sci. U.S.A., 85:2444, 1988; Higgins and Sharp, Gene, 73:237-244, 1988; Higgins and Sharp, CABIOS, 5:151-153, 1989; Corpet et al. Nuc. Acids Res., 16:10881-10890, 1988; Huang et al., Comp. Appls Biosci., 8:155-165, 1992; and Pearson et al., Meth. Mol. Biol., 24:307-31, 1994). Altschul et al., Nat. Genet., 6:119-129, 1994, presents a detailed consideration of sequence alignment methods and homology calculations. By way of example, the alignment tools ALIGN (Myers and Miller, CABIOS 4:11-17, 1989) or LFASTA (Pearson and Lipman, 1988) may be used to perform sequence comparisons (Internet Program® 1996, W. R. Pearson and the University of Virginia, fasta20u63 version 2.0u63, release date December 1996). ALIGN compares entire sequences against one another, while LFASTA compares regions of local similarity. These alignment tools and their respective tutorials are available on the Internet at the NCSA Website, for instance. Alternatively, for comparisons of amino acid sequences of greater than about 30 amino acids, the Blast 2 sequences function can be employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). The BLAST sequence comparison system is available, for instance, from the NCBI web site; see also Altschul et al., J. Mol. Biol., 215:403-410, 1990; Gish. & States, Nature Genet., 3:266-272, 1993; Madden et al. Meth. Enzymol., 266:131-141, 1996; Altschul et al., Nucleic Acids Res., 25:3389-3402, 1997; and Zhang & Madden, Genome Res., 7:649-656, 1997.

As used herein, the term “nucleic acid molecule” has its general meaning in the art and refers to a DNA or RNA molecule. However, the term captures sequences that include any of the known base analogues of DNA and RNA such as, but not limited to 4-acetylcytosine, 8-hydroxy-N6-methyladenosine, aziridinylcytosine, pseudoisocytosine, 5-(carboxyhydroxylmethyl) uracil, 5-fiuorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyamino-methyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, -uracil-5-oxyacetic acid methylester, uracil-5-oxyacetic acid, pseudouracil, queosine, 2-thiocytosine, and 2,6-diaminopurine.

In some embodiments, the nucleic acid molecule of the present invention comprises a sequence having at least 70% of identity with the nucleic acid sequence as set forth in SEQ ID NO:3, or SEQ ID NO:4.

According to the invention a first nucleic acid sequence having at least 70% of identity with a second nucleic acid sequence means that the first sequence has 70; 71; 72; 73; 74; 75; 76; 77; 78; 79; 80; 81; 82; 83; 84; 85; 86; 87; 88; 89; 90; 91; 92; 93; 94; 95; 96; 97; 98; 99 or 100% of identity with the second nucleic acid sequence.

SEQ ID NO: 3: codon-optimized nucleic acid
sequence encoding for the murine form of the
APPsα:
ttggaggtgcccaccgacggcaacgctggactgctggctgaaccccagat
cgccatgttctgcggcaagctgaacatgcacatgaacgtgcagaacggca
agtgggagagcgaccccagcggcaccaagacctgcatcggcaccaaagag
ggcatcctgcagtattgccaggaagtgtaccccgagctgcagatcaccaa
cgtggtggaagccaaccagcccgtgaccatccagaactggtgcaagaggg
gcagaaagcagtgcaagacccacacccacatcgtgatcccttacagatgc
ctcgtgggcgagttcgtgtccgacgctctgctggtgcccgacaagtgcaa
gttcctgcatcaggaacggatggacgtgtgcgagacacatctgcactggc
acaccgtggccaaagagacatgcagcgagaagtccaccaacctgcacgac
tacggcatgctgctgccctgcggcatcgacaagttcagaggcgtggaatt
cgtgtgctgccccctggccgaggaatccgactctgtggatagcgccgacg
ccgaagaggacgactctgacgtgtggtggggcggagccgacacagattac
gctgatggcggcgaggacaaggtggtggaagtggctgaagaggaagaggt
ggccgacgtggaagaagaagaggccgacgacgacgaggatgtggaagatg
gcgacgaggtggaagaggaagccgaggaaccctacgaggaagccaccgag
agaaccaccagcaccgccaccacaaccaccaccactaccgagagcgtgga
agaggtcgtgcgggtgccaacaacagccgcctctacacctgacgccgtgg
acaagtacctggaaaccccaggcgacgagaacgagcacgcccacttccag
aaggctaaagagagactggaagctaagcaccgcgagagaatgagccaagt
gatgagagagtgggaggaagctgagagacaggccaagaacctgcccaagg
ccgacaagaaagccgtgatccagcacttccaggaaaaggtggaaagcctg
gaacaggaagctgccaacgagagacagcagctggtggaaacccacatggc
cagagtggaagctatgctgaacgacagaagaaggctggccctggaaaact
acatcaccgctctgcaggccgtgccccccagacctcaccacgtgttcaac
atgctgaagaaatacgtgcgggccgagcagaaggacagacagcacaccct
gaagcacttcgagcacgtgcggatggtggaccccaagaaggccgcccaga
tcagatcccaagtgatgacccacctgagagtgatctacgagaggatgaac
cagagcctgagcctgctgtacaacgtgcccgccgtggccgaagaaatcca
ggatgaggtggacgagctgctgcagaaagagcagaactacagcgacgacg
tgctggccaacatgatcagcgagcccagaatcagctacggcaacgacgcc
ctgatgcccagcctgaccgagacaaagaccaccgtggaactgctgcccgt
gaacggcgagttcagcctggatgacctgcagccctggcaccattcggcgt
ggactctgtgcctgccaacacagagaacgaagtggaacccgtggacgcca
gacctgccgctgatagaggcctgaccacaagacctggcagcggcctgaca
aacatcaagaccgaagagatcagcgaagtgaagatggacgccgagttcgg
gcacgacagcggctttgaagtgcggcaccagaaa
SEQ ID NO: 4: codon-optimized nucleic acid
sequence encoding for the human form of the APPsα:
ttggaggtgcccaccgacggcaacgccggactgctggccgagccccagat
cgccatgttctgcggcagactgaacatgcacatgaacgtgcagaacggca
agtgggacagcgaccccagcggcaccaagacctgcatcgacaccaaagag
ggcatcctgcagtattgccaagaagtgtaccccgagctgcagatcaccaa
cgtggtggaagccaaccagcccgtgaccatccagaactggtgcaagcggg
gcagaaagcagtgcaagacccacccccacttcgtgatcccttacagatgc
ctcgtgggcgagttcgtgtccgacgccctgctggtgcccgacaagtgcaa
gttcctgcatcaagaacggatggacgtgtgcgagacacatctgcactggc
acaccgtggccaaagagacatgcagcgagaagtccaccaacctgcacgac
tacggcatgctgctgccctgcggcatcgacaagttccggggcgtggaatt
cgtgtgctgccccctggccgaggaatccgacaacgtggacagcgccgacg
ccgaagaggacgacagcgacgtgtggtggggcggagccgacaccgattac
gccgacggcagcgaggacaaggtggtggaagtggctgaagaggaagaggt
ggccgaggtcgaggaagaggaagccgacgacgacgaggatgacgaggacg
gcgacgaggtggaagaagaggccgaggaaccctacgaggaagccaccgag
cggaccacctctatcgccaccaccaccacaaccactaccgagagcgtgga
agaggtcgtgcgggtgccaaccaccgccgcctctacccccgacgccgtgg
acaagtacctggaaacccctggcgacgagaacgagcacgcccacttccag
aaggccaaagagcggctggaagccaagcaccgcgagcggatgagccaggt
catgagagagtgggaagaagccgagcggcaggccaagaacctgcccaagg
ccgacaagaaagccgtgatccagcacttccaagaaaaggtcgagagcctg
gaacaagaagccgccaacgagcggcagcagctggtggaaacccacatggc
cagagtggaagccatgctgaacgaccggcggagactggccctggaaaact
acatcaccgctctgcaggccgtgccccccagaccccggcacgtgttcaac
atgctgaagaaatacgtgcgggccgagcagaaggaccggcagcacaccct
gaagcacttcgagcacgtgcggatggtggaccccaagaaggccgcccaga
tccgctctcaggtcatgacccacctgagagtgatctacgagagaatgaac
cagagcctgagcctgctgtacaacgtgcccgccgtggccgaagaaatcca
ggatgaggtggacgagctgctgcagaaagagcagaactacagcgacgacg
tgctggccaacatgatcagcgagccccggatcagctacggcaacgacgcc
ctgatgcccagcctgaccgagacaaagaccaccgtggaactgctgcccgt
gaacggcgagttcagcctggacgacctgcagccctggcacagcttcggcg
ctgatagcgtgcccgccaacaccgagaatgaggtggaacccgtggacgcc
agacctgccgccgatagaggcctgaccacaagacctggcagcggcctgac
caacatcaagaccgaagagatcagcgaagtgaagatggacgccgagttcc
ggcacgacagcggctacgaggtgcaccaccagaaa

As used herein, the term “vector” has its general meaning in the art and refers to the vehicle by a nucleic acid molecule can be introduced into a host cell, so as to transform the host and promote expression (e.g. transcription and translation) of the introduced sequence. The terms “Gene transfer” or “gene delivery” refer to methods or systems for reliably inserting foreign DNA into host cells. Such methods can result in transient expression of non-integrated transferred DNA, extrachromosomal replication and expression of transferred replicons (e.g. episomes), or integration of transferred genetic material into the genomic DNA of host cells. Cells could be hematopoietic stem cells (e.g. CD34+ cell fraction) or hematopoietic progenitor cells (particularly monocytic progenitors or microglia precursors) isolated from the bone marrow or the blood of the patient (autologous) or from a donor (allogeneic) genetically modified to stably express APPsα or a fragment derived from it by transduction with a vector, particularly a lentiviral vector expressing APPsα under the control of a non-specific (e.g.: phosphoglycerate kinase, EF1alpha) or specific (monocytic-macrophage or microglia specific e.g. CD68 or CD11b) native or modified promoter.

In some embodiments, the vector of the present invention is a non-viral vector. Typically, the non-viral vector may be a plasmid which includes the nucleic acid molecule of the present invention.

In some embodiments, the vector of the present invention is a viral vector. Gene delivery viral vectors useful in the practice of the present invention can be constructed utilizing methodologies well known in the art of molecular biology. Typically, viral vectors carrying transgenes are assembled from polynucleotides encoding the transgene, suitable regulatory elements and elements necessary for production of viral proteins which mediate cell transduction. Examples of viral vector include but are not limited to adenoviral, retroviral, lentiviral, herpesvirus and adeno-associated virus (AAV) vectors.

In some embodiments, the vector of the present invention is an adeno-associated viral (AAV) vector. By an “AAV vector” is meant a vector derived from an adeno-associated virus serotype, including without limitation AAV1, AAV2, AAV3, AAV4, AA5, AAV6, AAV7, AAV8, AAV9, AAVrh10 or any other serotypes of AAV that can infect humans, monkeys or other species. AAV vectors can have one or more of the AAV wild-type genes deleted in whole or part, preferably the rep and/or cap genes, but retain functional flanking ITR sequences. Functional ITR sequences are necessary for the rescue, replication and packaging of the AAV virion. Thus, an AAV vector is defined herein to include at least those sequences required in cis for replication and packaging (e. g., functional ITRs) of the virus. The ITRs need not be the wild-type nucleotide sequences, and may be altered, e. g by the insertion, deletion or substitution of nucleotides, so long as the sequences provide for functional rescue, replication and packaging. AAV expression vectors are constructed using known techniques to at least provide as operatively linked components in the direction of transcription, control elements including a transcriptional initiation region, the nucleic acid molecule of the present invention and a transcriptional termination region. The control elements are selected to be functional in a mammalian cell. The resulting construct which contains the operatively linked components is bounded (5′ and 3′) with functional AAV ITR sequences. By “adeno-associated virus inverted terminal repeats” or “AAV ITRs” is meant the art-recognized regions found at each end of the AAV genome which function together in cis as origins of DNA replication and as packaging signals for the virus. AAV ITRs, together with the AAV rep coding region, provide for the efficient excision and rescue from, and integration of a nucleotide sequence interposed between two flanking ITRs into a mammalian cell genome. The nucleotide sequences of AAV ITR regions are known. See, e.g., Kotin, 1994; Berns, K I “Parvoviridae and their Replication” in Fundamental Virology, 2nd Edition, (B. N. Fields and D. M. Knipe, eds.) for the AAV-2 sequence. As used herein, an “AAV ITR” does not necessarily comprise the wild-type nucleotide sequence, but may be altered, e.g., by the insertion, deletion or substitution of nucleotides. Additionally, the AAV ITR may be derived from any of several AAV serotypes, including without limitation, AAV-1, AAV-2, AAV-3, AAV-4, AAV-5, AAV-6, etc. Furthermore, 5′ and 3′ ITRs which flank a selected nucleotide sequence in an AAV vector need not necessarily be identical or derived from the same AAV serotype or isolate, so long as they function as intended, i.e., to allow for excision and rescue of the sequence of interest from a host cell genome or vector, and to allow integration of the heterologous sequence into the recipient cell genome when AAV Rep gene products are present in the cell. Additionally, AAV ITRs may be derived from any of several AAV serotypes, including without limitation, AAV-1, AAV-2, AAV-3, AAV-4, AAV 5, AAV-6, etc. Furthermore, 5′ and 3′ ITRs which flank a selected nucleotide sequence in an AAV expression vector need not necessarily be identical or derived from the same AAV serotype or isolate, so long as they function as intended, i. e., to allow for excision and rescue of the sequence of interest from a host cell genome or vector, and to allow integration of the DNA molecule into the recipient cell genome when AAV Rep gene products are present in the cell. In some embodiments, the AAV vector of the present invention is selected from vectors derived from AAV serotypes having tropism for and high transduction efficiencies in cells of the mammalian central and peripheral nervous system, particularly neurons, neuronal progenitors, astrocytes, oligodendrocytes and glial cells. In some embodiments, the AAV vector is an AAV4, AAV9 or an AAVrh10 that have been described to well transduce brain cells especially neurons. In some embodiments, the AAV vector of the present invention is a double-stranded, self-complementary AAV (scAAV) vector. Alternatively to the use of single-stranded AAV vector, self-complementary vectors can be used. The efficiency of AAV vector in terms of the number of genome-containing particles required for transduction, is hindered by the need to convert the single-stranded DNA (ssDNA) genome into double-stranded DNA (dsDNA) prior to expression. This step can be circumvented through the use of self-complementary vectors, which package an inverted repeat genome that can fold into dsDNA without the requirement for DNA synthesis or base-pairing between multiple vector genomes. Resulting self-complementary AAV (scAAV) vectors have increased resulting expression of the transgene. For an overview of AAV biology, ITR function, and scAAV constructs, see McCarty D M. Self-complementary AAV vectors; advances and applications. Mol. Ther. 2008 October; 16 (10): at pages 1648-51, first full paragraph, incorporated herein by reference for disclosure of AAV and scAAV constructs, ITR function, and role of ATRS ITR in scAAV constructs. A rAAV vector comprising a ATRS ITR cannot correctly be nicked during the replication cycle and, accordingly, produces a self-complementary, double-stranded AAV (scAAV) genome, which can efficiently be packaged into infectious AAV particles. Various rAAV, ssAAV, and scAAV vectors, as well as the advantages and drawbacks of each class of vector for specific applications and methods of using such vectors in gene transfer applications are well known to those of skill in the art (see, for example, Choi V W, Samulski R J, McCarty D M. Effects of adeno-associated virus DNA hairpin structure on recombination. J. Virol. 2005 June; 79(11):6801-7; McCarty D M, Young S M Jr, Samulski R J. Integration of adeno-associated virus (AAV) and recombinant AAV vectors. Annu Rev Genet. 2004; 38:819-45; McCarty D M, Monahan P E, Samulski R J. Self-complementary recombinant adeno-associated virus (scAAV) vectors promote efficient transduction independently of DNA synthesis. Gene Ther. 2001 August; 8(16):1248-54; and McCarty D M. Self-complementary AAV vectors; advances and applications. Mol. Ther. 2008 October; 16(10):1648-56; all references cited in this application are incorporated herein by reference for disclosure of AAV, rAAV, and scAAV vectors).

The AAV vector of the present invention can be constructed by directly inserting the selected sequence (s) into an AAV genome which has had the major AAV open reading frames (“ORFs”) excised therefrom. Other portions of the AAV genome can also be deleted, so long as a sufficient portion of the ITRs remain to allow for replication and packaging functions. Such constructs can be designed using techniques well known in the art. See, e. g. U.S. Pat. Nos. 5,173,414 and 5,139,941; International Publications Nos. WO 92/01070 (published 23 Jan. 1992) and WO 93/03769 (published 4 Mar. 1993); Lebkowski et al., 1988; Vincent et al., 1990; Carter, 1992; Muzyczka, 1992; Kotin, 1994; Shelling and Smith, 1994; and Zhou et al., 1994. Alternatively, AAV ITRs can be excised from the viral genome or from an AAV vector containing the same and fused 5′ and 3′ of a selected nucleic acid construct that is present in another vector using standard ligation techniques. AAV vectors which contain ITRs have been described in, e. g. U.S. Pat. No. 5,139,941. In particular, several AAV vectors are described therein which are available from the American Type Culture Collection (“ATCC”) under Accession Numbers 53222, 53223, 53224, 53225 and 53226. Additionally, chimeric genes can be produced synthetically to include AAV ITR sequences arranged 5′ and 3′ of one or more selected nucleic acid sequences. Preferred codons for expression of the chimeric gene sequence in mammalian CNS and PNS cells can be used. The complete chimeric sequence is assembled from overlapping oligonucleotides prepared by standard methods. See, e. g., Edge, 1981; Nambair et al., 1984; Jay et al., 1984. In order to produce AAV virions, an AAV expression vector is introduced into a suitable host cell using known techniques, such as by transfection. A number of transfection techniques are generally known in the art. See, e. g., Graham et al., 1973; Sambrook et al. (1989) Molecular Cloning, a laboratory manual, Cold Spring Harbor Laboratories, New York, Davis et al. (1986) Basic Methods in Molecular Biology, Elsevier, and Chu et al., 1981. Particularly suitable transfection methods include calcium phosphate co-precipitation (Graham et al., 1973), direct microinjection into cultured cells (Capecchi, 1980), electroporation (Shigekawa et al., 1988), liposome mediated gene transfer (Mannino et al., 1988), lipid-mediated transduction (Felgner et al., 1987), and nucleic acid delivery using high-velocity microprojectiles (Klein et al., 1987).

Typically the vector of the present invention comprises an expression cassette. The term “expression cassette”, as used herein, refers to a nucleic acid construct comprising nucleic acid elements sufficient for the expression of the nucleic acid molecule of the present invention. Typically, an expression cassette comprises the nucleic acid molecule of the present invention operatively linked to a promoter sequence. The term “operatively linked” refers to the association of two or more nucleic acid fragments on a single nucleic acid fragment so that the function of one is affected by the other. For example, a promoter is operatively linked with a coding sequence when it is capable of affecting the expression of that coding sequence (e.g., the coding sequence is under the transcriptional control of the promoter). Encoding sequences can be operatively linked to regulatory sequences in sense or antisense orientation. In some embodiments, the promoter is a heterologous promoter. The term “heterologous promoter”, as used herein, refers to a promoter that is not found to be operatively linked to a given encoding sequence in nature. In some embodiments, an expression cassette may comprise additional elements, for example, an intron, an enhancer, a polyadenylation site, a woodchuck response element (WRE), and/or other elements known to affect expression levels of the encoding sequence. As used herein, the term “promoter” refers to a nucleotide sequence capable of controlling the expression of a coding sequence or functional RNA. In general, the nucleic acid molecule of the present invention is located 3′ of a promoter sequence. In some embodiments, a promoter sequence consists of proximal and more distal upstream elements and can comprise an enhancer element. An “enhancer” is a nucleotide sequence that can stimulate promoter activity and may be an innate element of the promoter or a heterologous element inserted to enhance the level or tissue-specificity of a promoter. In some embodiments, the promoter is derived in its entirety from a native gene. In some embodiments, the promoter is composed of different elements derived from different naturally occurring promoters. In some embodiments, the promoter comprises a synthetic nucleotide sequence. It will be understood by those skilled in the art that different promoters will direct the expression of a gene in different tissues or cell types, or at different stages of development, or in response to different environmental conditions or to the presence or the absence of a drug or transcriptional co-factor. Ubiquitous, cell-type-specific, tissue-specific, developmental stage-specific, and conditional promoters, for example, drug-responsive promoters (e.g. tetracycline-responsive promoters) are well known to those of skill in the art. Examples of promoter include, but are not limited to, the phophoglycerate kinase (PKG) promoter, CAG (composite of the CMV enhancer the chicken beta actin promoter (CBA) and the rabbit beta globin intron.), NSE (neuronal specific enolase), synapsin or NeuN promoters, the SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), SFFV promoter, rous sarcoma virus (RSV) promoter, synthetic promoters, hybrid promoters, and the like. The promoters can be of human origin or from other species, including from mice. In addition, sequences derived from non-viral genes, such as the murine metallothionein gene, will also find use herein. Such promoter sequences are commercially available from, e. g. Stratagene (San Diego, Calif.).

In some embodiments, the expression cassette comprises an appropriate secretory signal sequence that will allow the secretion of the polypeptide encoded by the nucleic acid molecule of the present invention. As used herein, the term “secretory signal sequence” or variations thereof are intended to refer to amino acid sequences that function to enhance (as defined above) secretion of an operably linked polypeptide from the cell as compared with the level of secretion seen with the native polypeptide. As defined above, by “enhanced” secretion, it is meant that the relative proportion of the polypeptide synthesized by the cell that is secreted from the cell is increased; it is not necessary that the absolute amount of secreted protein is also increased. In some embodiments, essentially all (i.e., at least 95%, 97%, 98%, 99% or more) of the polypeptide is secreted. It is not necessary, however, that essentially all or even most of the polypeptide is secreted, as long as the level of secretion is enhanced as compared with the native polypeptide. Generally, secretory signal sequences are cleaved within the endoplasmic reticulum and, in some embodiments, the secretory signal sequence is cleaved prior to secretion. It is not necessary, however, that the secretory signal sequence is cleaved as long as secretion of the polypeptide from the cell is enhanced and the polypeptide is functional. Thus, in some embodiments, the secretory signal sequence is partially or entirely retained. The secretory signal sequence can be derived in whole or in part from the secretory signal of a secreted polypeptide (i.e., from the precursor) and/or can be in whole or in part synthetic. The length of the secretory signal sequence is not critical; generally, known secretory signal sequences are from about 10-15 to 50-60 amino acids in length. Further, known secretory signals from secreted polypeptides can be altered or modified (e.g., by substitution, deletion, truncation or insertion of amino acids) as long as the resulting secretory signal sequence functions to enhance secretion of an operably linked polypeptide. The secretory signal sequences of the invention can comprise, consist essentially of or consist of a naturally occurring secretory signal sequence or a modification thereof (as described above). Numerous secreted proteins and sequences that direct secretion from the cell are known in the art. The secretory signal sequence of the invention can further be in whole or in part synthetic or artificial. Synthetic or artificial secretory signal peptides are known in the art, see e.g., Barash et al., “Human secretory signal peptide description by hidden Markov model and generation of a strong artificial signal peptide for secreted protein expression,” Biochem. Biophys. Res. Comm. 294:835-42 (2002); the disclosure of which is incorporated herein in its entirety.

In some embodiments, the vector of the present invention comprises the nucleic acid sequence set forth in SED ID NO:5 or 6.

SEQ ID NO: 5: complete sequence of the expression cassette of the AAV transfer
vector encoding codon-optimized mouse APPsα
ggggggggggggggggggttggccactccctctctgcgcgctcgctcgctcactgaggccgggcgaccaaaggtcgcc
cgacgcccgggctttgcccgggcggcctcagtgagcgagcgagcgcgcagagagggagtggccaactccatcactagg
ggttcctagatctaggatcacgcgttctagaaatattaaggtacgggaggtacttggagcggccgcaataaaatatct
ttattttcattacatctgtgtgttggttttttgtgtgaatcgatagtactaacatacgctctccatcaaaacaaaacg
aaacaaaacaaactagcaaaataggctgtccccagtgcaagtgggttttaggaccaggatgaggcggggtgggggtgc
ctacctgacgaccgaccccgacccactggacaagcacccaacccccattccccaaattgcgcatcccctatcagagag
ggggaggggaaacaggatgcggcgaggcgcgtgcgcactgccagcttcagcaccgcggacagtgccttcgcccccgcc
tggcggcgcgcgccaccgccgcctcagcactgaaggcgcgctgacgtcactcgccggtcccccgcaaactccccttcc
cggccaccttggtcgcgtccgcgccgccgccggcccagccggaccgcaccacgcgaggcgcgagataggggggcacgg
gcgcgaccatctgcgctgcggcgccggcgactcagcgctgcctcagtctgcggtgggcagcggaggagtcgtgtcgtg
cctgagagcgcagtcgaattgctagcggggatccaccggtcgccaccatgctgccttctctggctttgctgctgctgg
ccgcttggacagtgcgggcctacccttacgacgtgcccgactacgcttacccctacgatgtgcctgattatgcattgg
aggtgcccaccgacggcaacgctggactgctggctgaaccccagatcgccatgttctgcggcaagctgaacatgcaca
tgaacgtgcagaacggcaagtgggagagcgaccccagcggcaccaagacctgcatcggcaccaaagagggcatcctgc
agtattgccaggaagtgtaccccgagctgcagatcaccaacgtggtggaagccaaccagcccgtgaccatccagaact
ggtgcaagaggggcagaaagcagtgcaagacccacacccacatcgtgatcccttacagatgcctcgtgggcgagttcg
tgtccgacgctctgctggtgcccgacaagtgcaagttcctgcatcaggaacggatggacgtgtgcgagacacatctgc
actggcacaccgtggccaaagagacatgcagcgagaagtccaccaacctgcacgactacggcatgctgctgccctgcg
gcatcgacaagttcagaggcgtggaattcgtgtgctgccccctggccgaggaatccgactctgtggatagcgccgacg
ccgaagaggacgactctgacgtgtggtggggcggagccgacacagattacgctgatggcggcgaggacaaggtggtgg
aagtggctgaagaggaagaggtggccgacgtggaagaagaagaggccgacgacgacgaggatgtggaagatggcgacg
aggtggaagaggaagccgaggaaccctacgaggaagccaccgagagaaccaccagcaccgccaccacaaccaccacca
ctaccgagagcgtggaagaggtcgtgcgggtgccaacaacagccgcctctacacctgacgccgtggacaagtacctgg
aaaccccaggcgacgagaacgagcacgcccacttccagaaggctaaagagagactggaagctaagcaccgcgagagaa
tgagccaagtgatgagagagtgggaggaagctgagagacaggccaagaacctgcccaaggccgacaagaaagccgtga
tccagcacttccaggaaaaggtggaaagcctggaacaggaagctgccaacgagagacagcagctggtggaaacccaca
tggccagagtggaagctatgctgaacgacagaagaaggctggccctggaaaactacatcaccgctctgcaggccgtgc
cccccagacctcaccacgtgttcaacatgctgaagaaatacgtgcgggccgagcagaaggacagacagcacaccctga
agcacttcgagcacgtgcggatggtggaccccaagaaggccgcccagatcagatcccaagtgatgacccacctgagag
tgatctacgagaggatgaaccagagcctgagcctgctgtacaacgtgcccgccgtggccgaagaaatccaggatgagg
tggacgagctgctgcagaaagagcagaactacagcgacgacgtgctggccaacatgatcagcgagcccagaatcagct
acggcaacgacgccctgatgcccagcctgaccgagacaaagaccaccgtggaactgctgcccgtgaacggcgagttca
gcctggatgacctgcagccctggcaccctttcggcgtggactctgtgcctgccaacacagagaacgaagtggaacccg
tggacgccagacctgccgctgatagaggcctgaccacaagacctggcagcggcctgacaaacatcaagaccgaagaga
tcagcgaagtgaagatggacgccgagttcgggcacgacagcggctttgaagtgcggcaccagaaatagaagcttatcg
ataatcaacctctggattacaaaatttgtgaaagattgactggtattcttaactatgttgctccttttacgctatgtg
gatacgctgctttaatgcctttgtatcatgctattgcttcccgtatggctttcattttctcctccttgtataaatcct
ggttgctgtctctttatgaggagttgtggcccgttgtcaggcaacgtggcgtggtgtgcactgtgtttgctgacgcaa
cccccactggttggggcattgccaccacctgtcagctcctttccgggactttcgctttccccctccctattgccacgg
cggaactcatcgccgcctgccttgcccgctgctggacaggggctcggctgttgggcactgacaattccgtggtgttgt
cggggaagctgacgtcctttccatggctgctcgcctgtgttgccacctggattctgcgcgggacgtccttctgctacg
tcccttcggccctcaatccagcggaccttccttcccgcggcctgctgccggctctgcggcctcttccgcgtcttcgcc
ttcgccctcagacgagtcggatctccctttgggccgcctccccgcctgatcgataccgtcgactagagctcgctgatc
agcctcgactgtgccttctagttgccagccatctgttgtttgcccctcccccgtgccttccttgaccctggaaggtgc
cactcccactgtcctttcctaataaaatgaggaaattgcatcgcattgtctgagtaggtgtcattctattctgggggg
tggggtggggcaggacagcaagggggaggattgggaagacaatagcaggcatgctggggagagatctgaggaacccct
agtgatggagttggccactccctctctgcgcgctcgctcgctcactgaggccgcccgggcaaagcccgggcgtcgggc
gacctttggtcgcccggcctcagtgagcgagcgagcgcgcagagagggagtggccaaccccccccccccccccc
SEQ ID NO: 6: complete sequence of the expression cassette of the AAV transfer
vector encoding codon-optimized human APPsα
Ggggggggggggggggggttggccactccctctctgcgcgctcgctcgctcactgaggccgggcgaccaaaggtcgcc
cgacgcccgggctttgcccgggcggcctcagtgagcgagcgagcgcgcagagagggagtggccaactccatcactagg
ggttcctagatctaggatcacgcgttctagaaatattaaggtacgggaggtacttggagcggccgcaataaaatatct
ttattttcattacatctgtgtgttggttttttgtgtgaatcgatagtactaacatacgctctccatcaaaacaaaacg
aaacaaaacaaactagcaaaataggctgtccccagtgcaagtgggttttaggaccaggatgaggcggggtgggggtgc
ctacctgacgaccgaccccgacccactggacaagcacccaacccccattccccaaattgcgcatcccctatcagagag
ggggaggggaaacaggatgcggcgaggcgcgtgcgcactgccagcttcagcaccgcggacagtgccttcgcccccgcc
tggcggcgcgcgccaccgccgcctcagcactgaaggcgcgctgacgtcactcgccggtcccccgcaaactccccttcc
cggccaccttggtcgcgtccgcgccgccgccggcccagccggaccgcaccacgcgaggcgcgagataggggggcacgg
gcgcgaccatctgcgctgcggcgccggcgactcagcgctgcctcagtctgcggtgggcagcggaggagtcgtgtcgtg
cctgagagcgcagtcgaattgctagcggggatccaccggtcgccaccatgctgcctggactggctctgctgctgctgg
ccgcctggacagccagagcctacccctacgacgtgcccgactacgcctacccttatgatgtgcctgactatgcattgg
aggtgcccaccgacggcaacgccggactgctggccgagccccagatcgccatgttctgcggcagactgaacatgcaca
tgaacgtgcagaacggcaagtgggacagcgaccccagcggcaccaagacctgcatcgacaccaaagagggcatcctgc
agtattgccaagaagtgtaccccgagctgcagatcaccaacgtggtggaagccaaccagcccgtgaccatccagaact
ggtgcaagcggggcagaaagcagtgcaagacccacccccacttcgtgatcccttacagatgcctcgtgggcgagttcg
tgtccgacgccctgctggtgcccgacaagtgcaagttcctgcatcaagaacggatggacgtgtgcgagacacatctgc
actggcacaccgtggccaaagagacatgcagcgagaagtccaccaacctgcacgactacggcatgctgctgccctgcg
gcatcgacaagttccggggcgtggaattcgtgtgctgccccctggccgaggaatccgacaacgtggacagcgccgacg
ccgaagaggacgacagcgacgtgtggtggggcggagccgacaccgattacgccgacggcagcgaggacaaggtggtgg
aagtggctgaagaggaagaggtggccgaggtcgaggaagaggaagccgacgacgacgaggatgacgaggacggcgacg
aggtggaagaagaggccgaggaaccctacgaggaagccaccgagcggaccacctctatcgccaccaccaccacaacca
ctaccgagagcgtggaagaggtcgtgcgggtgccaaccaccgccgcctctacccccgacgccgtggacaagtacctgg
aaacccctggcgacgagaacgagcacgcccacttccagaaggccaaagagcggctggaagccaagcaccgcgagcgga
tgagccaggtcatgagagagtgggaagaagccgagcggcaggccaagaacctgcccaaggccgacaagaaagccgtga
tccagcacttccaagaaaaggtcgagagcctggaacaagaagccgccaacgagcggcagcagctggtggaaacccaca
tggccagagtggaagccatgctgaacgaccggcggagactggccctggaaaactacatcaccgctctgcaggccgtgc
cccccagaccccggcacgtgttcaacatgctgaagaaatacgtgcgggccgagcagaaggaccggcagcacaccctga
agcacttcgagcacgtgcggatggtggaccccaagaaggccgcccagatccgctctcaggtcatgacccacctgagag
tgatctacgagagaatgaaccagagcctgagcctgctgtacaacgtgcccgccgtggccgaagaaatccaggatgagg
tggacgagctgctgcagaaagagcagaactacagcgacgacgtgctggccaacatgatcagcgagccccggatcagct
acggcaacgacgccctgatgcccagcctgaccgagacaaagaccaccgtggaactgctgcccgtgaacggcgagttca
gcctggacgacctgcagccctggcacagcttcggcgctgatagcgtgcccgccaacaccgagaatgaggtggaacccg
tggacgccagacctgccgccgatagaggcctgaccacaagacctggcagcggcctgaccaacatcaagaccgaagaga
tcagcgaagtgaagatggacgccgagttccggcacgacagcggctacgaggtgcaccaccagaaatagaagcttatcg
ataatcaacctctggattacaaaatttgtgaaagattgactggtattcttaactatgttgctccttttacgctatgtg
gatacgctgctttaatgcctttgtatcatgctattgcttcccgtatggctttcattttctcctccttgtataaatcct
ggttgctgtctctttatgaggagttgtggcccgttgtcaggcaacgtggcgtggtgtgcactgtgtttgctgacgcaa
cccccactggttggggcattgccaccacctgtcagctcctttccgggactttcgctttccccctccctattgccacgg
cggaactcatcgccgcctgccttgcccgctgctggacaggggctcggctgttgggcactgacaattccgtggtgttgt
cggggaagctgacgtcctttccatggctgctcgcctgtgttgccacctggattctgcgcgggacgtccttctgctacg
tcccttcggccctcaatccagcggaccttccttcccgcggcctgctgccggctctgcggcctcttccgcgtcttcgcc
ttcgccctcagacgagtcggatctccctttgggccgcctccccgcctgatcgataccgtcgactagagctcgctgatc
agcctcgactgtgccttctagttgccagccatctgttgtttgcccctcccccgtgccttccttgaccctggaaggtgc
cactcccactgtcctttcctaataaaatgaggaaattgcatcgcattgtctgagtaggtgtcattctattctgggggg
tggggtggggcaggacagcaagggggaggattgggaagacaatagcaggcatgctggggagagatctgaggaacccct
agtgatggagttggccactccctctctgcgcgctcgctcgctcactgaggccgcccgggcaaagcccgggcgtcgggc
gacctttggtcgcccggcctcagtgagcgagcgagcgcgcagagagggagtggccaaccccccccccccccccc

By a “therapeutically effective amount” of the vector of the present invention is meant a sufficient amount of the vector for the treatment of Down syndrome and Alzheimer's disease. It will be understood, however, that the total daily usage of the vector of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific vector employed; and like factors well known in the medical arts. For example, it is well within the skill of the art to start doses of the compound at levels lower than those required to achieve the desired therapeutic effect and to gradually increase the dosage until the desired effect is achieved. Typically, from 10⁸ to 10¹⁰ viral genomes (vg) are administered per dose in mice. Typically, the doses of AAV vectors to be administered in humans may range from 10¹⁰ to 10¹² vg.

Typically, the vector or the cell of the present invention are delivered directly and specifically into selected brain regions by intracerebral injections into the cerebellum, the dentate nucleus, the striatum, the cortex and particularly the entorhinal cortex, or the hippocampus. In some embodiments, the vector of the present invention or the cells transduced with the vector are delivered by intrathecal delivery. In some embodiments, the vector of the present invention of the cells are delivered into the brain by intracerebral injection and/blood by intravenously injection, in the spinal fluid by intrathecal delivery, by or intracerebroventricular injection or by intra-nasal injection. Particularly, any routes of administration that allow a strong expression of the vector in the spinal cord, brain, cortex, hippocampus, and dentate nucleus can be used in the invention. In some embodiments, the cells are delivered by infusion in the peripheral blood (intravenous or intra-arterial injection) or in the CSF.

In some embodiments, the vector of the present invention is administrated to the subject in need thereof one time, two times, three times or more. In some embodiments, the vector of the present invention is administrated to the subject in need thereof one time and re-administered several months or years later to said subject.

The vectors used herein may be formulated in any suitable vehicle for delivery. For instance they may be placed into a pharmaceutically acceptable suspension, solution or emulsion. Suitable mediums include saline and liposomal preparations. More specifically, pharmaceutically acceptable carriers may include sterile aqueous of non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents, and inert gases and the like. A colloidal dispersion system may also be used for targeted gene delivery. Colloidal dispersion systems include macromolecule complexes, nanocapsules, microspheres, beads, and lipid-based systems including oil-in-water emulsions, micelles, mixed micelles, exosomes and liposomes.

In another aspect, the present invention relates to a method of treating Alzheimer's disease in a subject in need thereof comprising administering to the subject a therapeutically effective amount of cells transduced with a vector which comprises a nucleic acid molecule encoding for a polypeptide which is a soluble member of the APP (amyloid precursor protein) family.

In one embodiment, the cells administrated according to the invention are autologous hematopoietic stem cell or hematopoietic progenitors that could be isolated from the patient, transduced with a vector, particularly a lentiviral vector and reinfused directly or after bone marrow conditioning.

The invention will be further illustrated by the following figures and examples. However, these examples and figures should not be interpreted in any way as limiting the scope of the present invention.

### FIGURES

FIG. 1: APPsα overexpression enhances Morris water maze performance in WT mice and rescues the spatial memory deficit of APP/PS1ΔE9 mice. Transgenic APP/PS1ΔE9 mice (n=8 per group) or littermate (LM) controls (n=3-4 per group) were either injected with AAV-Venus or AAV-APPsα vectors at 12 months of age and tested 2 months later at 14 months of age. (A) Escape latency and (B) swim speed of littermate controls or APP/PS1ΔE9 mice injected either with AAV-Venus or AAV-APPsα. Swim speed was similar between the different groups (2-way ANOVA: Group effect: F_{3,100}=2.40; ns; Time effect: F_{4,100}=1.41; ns; Group×Time interaction: F_{12,100}<1; ns). Littermates injected with AAV-APPsα showed improved performance, as indicated by reduced escape latency (2-way ANOVA: Time effect: F_{4,100}=7.138; p<0.0001; Group effect: F_{3,100}=7.247; p=0.0002, followed by Tukey post-hoc test: APP/PS1ΔE9 mice injected with AAV-APPsα versus each of the other groups, p<0.013). (C) Probe trial performance at 72 h. 2-way ANOVA, Group effect: F_{3,17}=3.356; p<0.04; quadrant effect: F_{1,17}=23.54; p<0.007; Group×quadrant interaction effect: F_{3,17}=3.356; p<0.04. APP/PS1ΔE9 mice injected with AAV-Venus were impaired in comparison to littermate mice injected with AAV-Venus (Tukey post-hoc test: p=0.023) confirmed by no preference for the trained target quadrant. Strikingly, AAV-APPsα treated APP/PS1ΔE9 mice spent more time in the target quadrant compared to APP/PS1ΔE9 mice injected with AAV-Venus (Tukey post-hoc test: p=0.017). Statistics: 2-way ANOVA (genotype and group as factors) with repeated measures followed by Tukey post-hoc test: *<0.05. Values represent means±SEM.

FIG. 2: APP/PS1ΔE9 mice reveal structural and functional synaptic impairments that are ameliorated by APPsα expression. (A) LTP was induced by TBS at hippocampal CA3-CA1 synapses after 20 min baseline recordings. Acute slices of AAV-Venus injected APP/PS1ΔE9 animals exhibited significant lower induction and maintenance of LTP compared to littermate controls (LM) showing similar expression of Venus (averaged potentiation minutes t50-80: 148.47±6.04% vs. 178.01±8.98%, p=0.021). Viral expression of APPsα restored potentiation after TBS (171.48±6.29%) in transgenic animals and resulted in an LTP curve progression comparable to that of LM controls. The LTP induction rate is shown as percentage % of mean baseline slope, n=number of slices, N=number of mice. (B, C) Input-Output strength of all AAV-injected mice showed no alterations between genotypes at any fiber volley (FV) amplitude or stimulus intensity tested. (D) Altered PPF at the 10 ms ISI revealed a significant impairment in the pre-synapse of APP/PS1ΔE9 mice injected with AAV-Venus in comparison to LM controls (*p=0.030) that was restored after AAV-APPsα injection (#p=0.047). (E) No differences in spine density at CA1 apical neurons between groups, but significantly less spines at basal dendrites of APP/PS1ΔE9 Venus injected mice (p=0.040). APPsα overexpression partially restored the spine density deficit. (F) Reduced spine density at CA3 apical (p=0.014) and basal (p=0.011) dendritic segments of APP/PS1ΔE9 AAV-Venus injected mice that is partially rescued at apical and completely at basal dendrites (p=0.039) by APPsα. N=number of neurons, N=number of animals. Data represent mean±SEM and were analyzed by one-way ANOVA followed by Bonferroni's post-hoc test.

FIG. 3: AAV-APPsα injection decreases Aβ and plaques load. (A) ELISA quantification of β-CTF in hippocampus (H) and cortex (Cx) of APP/PS1ΔE9 mice. No difference was detectable between AAV-Venus and AAV-APPsα injected animals. (B-D) Quantification (MSD immunoassay) of TBS soluble Aβ38 (Group effect: F_{1,14}=3.879, p=0.07 (−36%), Aβ40 (Group effect: F_{1,14}=3.094, p=0.10 (−32%)) and Aβ42 (Group effect: F_{1,14}=5.211, p=0.04 (−33%); region effect: F_{1,14}<1, ns; Group×region interaction effect: F_{1,14}<1, ns) in hippocampus and cortex of APP/PS1ΔE9 mice. Note that AAV-APPsα injected animals show reduced levels of Aβ in both anatomical regions analyzed (hippocampus and cortex). (E) Quantification of 4G8 immunolabeled area in hippocampus and cortex (2-way ANOVA: Treatment effect: F_{1,13}=5.50, p=0.04 (−24%); region effect: F_{1,13}=22.89, p=0.0004; Treatment×region interaction effect: F_{1,13}<1, ns). Note that 4G8 immunoreactive plaque area is significantly reduced in AAV-APPsα treated animals. Number of animals n=8/group. 2-way ANOVA (Genotype, treatment and region as factors) followed by Tukey post-hoc test: *p<0.05. Values represent means±SEM.

FIG. 4: AAV-APPsα promotes microglia recruitment around plaques in APP/PS1ΔE9 mice. (A) Quantification signal intensity from western blot analysis showing the expression of GFAP and Iba1 in the hippocampus of AAV-Venus or AAV-APPsα injected APP/PS1ΔE9 mice (n=8/group). Quantification signal intensities were normalized to GAPDH used as a loading control. AAV-APPsα treatment specifically increased Iba1 (microglial marker) expression (t-test: t₁₄=3.586; p=0.003), whereas the astrocyte marker GFAP was not affected. (B) Whereas the distribution of GFAP positive astrocytes is unaltered, increased recruitment of Iba1 positive microglia is observed in the vicinity of amyloid plaques (t-test: t₁₆=5.441; p<0.0001). (C) Western blot analysis showing the expression of IDE and TREM2 in the hippocampus of AAV-Venus or AAV-APPsα injected APP/PS1ΔE9 mice. Both Aβ clearance related proteins are significantly upregulated (IDE: t-test: t₁₄=3.984; p=0.0014; TREM2: t-test: t₁₄=2.947; p=0.010) following AAV-APPsα treatment. Values represent means±SEM. ***P<0.001, **P<0.01, *P<0.05.

FIG. 5: Unlike APPsα, APPsβ overexpression does not rescue spatial memory deficit of APP/PS1ΔE9 mice in the Morris water maze. Littermate (LM) controls injected with Venus (n=3) or transgenic APP/PS1ΔE9 mice (n=7-8 per group) either injected with AAV-Venus, AAV-APPsα or AAV-APPsβ vectors at 12 months of age were tested 2 months later at 14 months of age. Graphic showing the probe trial performance at 72 h. 2-way ANOVA, group×quadrant interaction effect: F3,19=4.04; p=0.02. APP/PS1ΔE9 mice injected with AAV-Venus were impaired in comparison to littermate mice injected with AAV-Venus (Tukey post hoc test: p=0.04) confirmed by no preference for the trained target quadrant. Unlike AAV-APPsα treatment in APP/PS1ΔE9 mice which restored time spent in the target quadrant (Tukey post hoc test: p=0.011), AAV-APPsβ did not improve their performances compared to APP/PS1ΔE9 mice injected with AAV-Venus (Tukey post hoc test: p>0.99). Data represent mean±SEM and were analyzed by 2-way ANOVA (genotype and group as factors) with repeated measures followed by Tukey post hoc test. *p<0.05.

FIG. 6: AAV-APPsβ injection do not restore long term potentiation in the hippocampus of aged APP/PS1ΔE9 mice. LTP was induced by TBS at hippocampal CA3-CA1 synapses after 20 min baseline recordings. Acute slices of AAV-Venus injected APP/PS1ΔE9 animals exhibited significant lower induction and maintenance of LTP compared to littermate controls (LM) indicating a significant impairment of the transgenic mice. Viral expression of APPsα restored potentiation after TBS in transgenic animals and resulted in an LTP comparable to that of LM controls. However, AAV-APPsβ injection did not restore APP/PS1ΔE9 mice which show a similar level compared to AAV-Venus transgenic mice. The LTP induction rate is shown as percentage % of mean baseline slope, n=number of slices, N=number of mice.

FIG. 7: AAV-APPsβ injection does not activate microglia. A Western blot analysis showing the expression of IBA1 (microglial marker) in the hippocampus of AAV-Venus, AAVAPPsα or AAV-APPsβ injected APP/PS1ΔE9 mice (n=7/8 per group). For quantification signal intensity was normalized to GAPDH used as a loading control (One-way ANOVA: group effect: F2,19=14.38, p=0.0002). AAV-APPsα treatment specifically increased IBA1 expression (p=0.0002) whereas AAV-APPsβ did not (p=0.82). B Quantification of IBA1 signal in the hippocampus following immunohistochemistry in APP/PS1ΔE9 mice injected either with AAV-Venus, AAV-APPsα or AAV-APPsβ (One-way ANOVA: group effect: F2,41=14.12, p<0.0001). As seen in western blot, AAV-APPsβ injection is unable to rise IBA1 levels compared to AAV-Venus treated mice. Values represent mean±SEM. ***p<0.001, **p<0.01.

### EXAMPLE

Material & Methods

AAV Plasmid Design and Vector Production

The mouse APPsα coding sequence (derived from Uniprot: P12023-2) was codon optimized (Geneart, Regensburg) and then cloned under control of the synapsin promoter into the single stranded, rAAV2-based transfer vector pAAVSynMCS-2A-Venus (Tang et al, 2009) via NheI-HindIII restriction sites. For easy detection, an N-terminal double HA-tag was inserted downstream of the APP signal peptide at the N-terminus of APPsα. The control vector (pAAV-Venus) encodes the yellow fluorescent protein Venus fused to a C-terminal farnesylation signal for membrane anchoring. All constructs were packaged into AAV9 by the MIRCen viral production platform as described (Berger et al, 2015).

Animals

Sixteen APPswe/PS1ΔE9 mice (referred as APP/PS1ΔE9; Jackson Laboratories) and seven age-matched littermate control mice were used for behavior, pathology and biochemistry. Eleven APP/PS1ΔE9 and five littermates were used for electrophysiology and spine density analysis. APP/PS1ΔE9 mice express the human APP gene carrying the Swedish double mutation (K595N/M596L). In addition, they express the human PS1ΔE9 variant lacking exon 9 (Borchelt et al, 1997; Jankowsky et al, 2004; Xiong et al, 2011). Only male mice were used throughout the study. For age at AAV injection and age at analysis/sacrifice see results section. All experiments were conducted in accordance with the ethical standards of French, German and European regulations (European Communities Council Directive of 24 Nov. 1986).

Stereotactic Injection of AAV

Mice were anesthetized by intraperitoneal injection of ketamine/xylazine (0.1/0.05 g/kg body weight) and positioned on a stereotactic frame (Stoelting, Wood Dale, USA). Vectors (either AAV-Venus or AAV-APPsα) were bilaterally injected into the hippocampus using 2 μl of viral preparation (10¹⁰ vg/site) at a rate of 0.2 μl/minute. Two injections sites per hippocampus were used to optimize virus spreading. Stereotactic coordinates of injection sites from bregma were: anteroposterior −2 mm; mediolateral+/−1 mm; dorsoventral −2.25 mm and anteroposterior −2 mm; mediolateral+/−1 mm; dorsoventral −1.75 mm.

Brain Samples

APP/PS1ΔE9 mice were sacrificed 5 months post-injection at 17 months of age. Following anesthesia, mice were transcardially perfused with 0.1 M phosphate buffered saline (PBS) before dissection. For immunohistochemistry, the left cerebral hemisphere was dissected and post-fixed in 4% paraformaldehyde (PFA) for 1 week and cryoprotected in 30% sucrose for 24 hours. 40 μm sections were cut using a freezing microtome (Leica, Wetzlar, Germany), collected in a cryoprotective solution and stored at −20° C. The right hemisphere was dissected to segregate hippocampus and cortex for biochemical analysis. Samples were then homogenized in lysis buffer (TBS, NaCl 150 mM and Triton 1%) containing phosphatase and protease inhibitors. After centrifugation (20 min, 13 000 rpm, 4° C.), the supernatant was collected and the protein concentration was quantified by BCA Assay (Thermo Fisher Scientific, Waltham, USA). Lysate aliquots (3 mg of protein/ml) were stored at −80° C.

Immunostaining

Slices were washed with 0.1 M PBS and permeabilized in 0.25% PBS-Triton before blocking in PBS-Triton 0.25% containing 5% goat serum for 60 minutes. For vector encoded HA-APPsα immuno labeling, slices were incubated with an anti-HA antibody (Covance, Princeton, USA, 1/250) overnight at 4° C. After successive washes (PBS-Triton 0.25%, PBS and PB 0.1 M), incubation with a biotinylated anti-mouse antibody was performed for one hour at room temperature. For signal amplification, samples were incubated using the ABC kit (Vector laboratories, Burlingame, USA) for one hour at room temperature. Finally, slices were incubated in Cy3-coupled streptavidine. HA-APPsα was co-immunostained overnight with the following primary antibodies: Rabbit anti-Iba1, 1/500, Wako, Richmond, USA; Mouse-GFAP Cy3 conjugate, 1/500, Sigma-Aldrich, Saint-Louis, USA. For immunofluorescent staining of plaques, slices were stained using a 30 min incubation in 1% thioflavin-S solution, rinsed twice (1 min each) in 50% EtOH and mounted in Vectashield fluorescent mounting media (Vector laboratories). Images were taken with a Nikon Eclipse Ti microscope (Nikon, Tokyo, Japan) and a Leica SP8 confocal microscope (Leica). For plaque quantification, slices were incubated in 88% formic acid solution for 15 min (antigen retrieval). To inactivate endogenous peroxidase, samples were incubated in hydrogen peroxide (30 min) before blocking and incubation with the primary antibody (4G8, Covance, 1/1000). Incubation with a horseradish coupled secondary antibody was done at RT, developed using the DAB kit (Vector laboratories) and mounted in Eukitt mounting media (Sigma-Aldrich, Saint-Louis, USA). Images were taken with a Z6 APO macroscope (Leica). Plaques, GFAP and Iba1 immunoreactivity were quantified using ImageJ (NIH, Bethesda, USA) or Icy (Institut Pasteur, Paris, France). Laserpower, numeric gain and magnification were kept constant between animals to avoid potential technical artefacts. Images were first converted to 8-bit gray scale and binary thresholded to highlight a positive staining. At least 2 sections per mouse (between −1.7 mm to −2.3 mm caudal to bregma) were quantified for either hippocampus or cortex. The average value per structure was calculated for each mouse. For quantification of Iba1 and GFAP immunoreactivity around plaques, a region of interest (ROI) was drawn around the center of the plaque. The diameter of the circular ROI was set as 3 times the diameter of the plaque. Mean fluorescence intensity values were measured for either Iba1 or GFAP immunoreactivity and were processed via Icy software (Institut Pasteur, Paris, France). Experimentators and data managers were blind with respect to treatments and genotypes.

Western Blot Analysis

Proteins were separated by electrophoresis using 4-12% SDS-PAGE (NuPAGE, Life Technologies, Carlsbad, USA) in MOPS buffer (NuPAGE, Life Technologies) and transferred to nitrocellulose membranes (iBlot, Life Technologies). After blocking in 5% milk-PBS 0.1M for 60 minutes, membranes were incubated with the primary antibodies overnight at 4° C. (HA, 1/2000, Covance, Princeton, USA; Venus (GFP), 1/1000, Vector laboratories Burlingame, USA; GAPDH, 1/4000 Abcam, Cambridge, UK; Iba1, 1/2000, Wako, Richmond, USA; GFAP, 1/4000 Dako, Glostrup, Denmark; IDE, 1/200, Santa Cruz Biotechnology, Dallas, USA; TREM2, 1/500, R&D Systems, Minneapolis, USA). Membranes were then washed with TBS-T (with 0.1% Tween), incubated with a horseradish peroxidase coupled secondary antibody and developed using enhanced chemiluminescence (ECL, GE Healthcare, Little Chalfont, UK and Super Signal, Thermo Fisher Scientific). Signals were detected with Fusion FX7 (Vilber Lourmat, Marne-la-Vallée, France) and analyzed and quantified using ImageJ.

ELISA

APPsα, β-CTF, and Aβ were quantified using the sAPPα kit (Meso Scale Discovery, Rockville, USA), Human APP β-CTF Assay Kit (IBL, Hamburg, Germany), V-PLEX Plus Aβ Peptide Panel 1 (6E10) Kit (Meso Scale Discovery). The procedures were performed according to the respective supplier instructions.

Morris Water Maze

Experiments were performed in a 120-cm diameter, 50 cm deep tank filled with opacified water kept at 21° C. and equipped with a 10 cm diameter platform submerged 1 cm under the water surface. Visual clues were disposed around the pool as spatial landmarks for the mouse and luminosity was kept at 430 lux. Training consisted of daily sessions (three trials per session) during 5 consecutive days. Start positions varied pseudo-randomly among the four cardinal points. Mean inter-trial interval was 15 min. Each trial ended when the animal reached the platform. A 60 second cut-off was used, after which mice were gently guided to the platform. Once on the platform, animals were given a 30-second rest before being returned to their cage. 72 hours after the last training trial (day 8), retention was assessed during probe trial in which the platform was no longer present. Animals were video tracked using Ethovision software (Noldus, Wageningen, Netherlands) and behavioral parameters (swim speed, travelled distance, latency, percentage of time spend in each quadrant) were automatically calculated. Experiments and statistical evaluation of data were performed by an experimentator blind to genotype and treatment group.

Statistics

Statistical analyses were performed as indicated for the respective experiments. Outliers were detected and rejected using maximum normed residual test (Grubbs' test). In most cases, data were analyzed using non-parametric Mann-Whitney U tests excepted for behavioral experiments. Two-way ANOVA with repeated measures were carried out when required by the experimental plan to assess statistical effects. Correlation matrices were generated using non-parametric Spearman rank correlation coefficient. For all analysis statistical significance was set to a p-value <0.05. All analyses were performed using Statistica (StatSoft Inc., Tulsa, USA) or Prism (GraphPad Software, La Jolla, USA).

Electrophysiology

In vitro extracellular recordings were performed on acute hippocampal slices of WT littermates stereotactically injected with the AAV-Venus (N=5), APP/PS1ΔE9 mice injected either with AAV-Venus (N=4) or AAV-APPsα virus (N=6) at 8 months of age. Electrophysiological recordings were performed 4-5 months later at an age of 12-13 months. In-between animals were housed in a temperature- and humidity-controlled room with a 12 h light-dark cycle and had access to food and water ad libitum.

Slice Preparation

Acute hippocampal transversal slices were prepared from individuals at an age of 12 to 13 months. Mice were anesthetized with isoflurane and decapitated. The brain was removed and quickly transferred into ice-cold carbogenated (95% O₂, 5% CO₂) artificial cerebrospinal fluid (ACSF) containing 125 mM NaCl, 2 mM KCl, 1.25 mM NaH₂PO₄, 2 mM MgCl₂, 26 mM NaHCO₃, 25 mM glucose. After dissection of the two hemispheres one was used for Golgi-Cox staining and the other for electrophysiology. The hippocampus was sectioned into 400 μm thick transversal slices with a vibrating microtome (Leica, VT1200S). Slices were maintained in carbogenated ACSF (125 mM NaCl, 2.mM KCl, 1.25 mM NaH₂PO₄, 2 mM MgCl₂, 26 mM NaHCO₃, 2 mM CaCl₂, 25 mM glucose) at room temperature for at least 1.5 h before transferred into a submerged recording chamber. Before recording, each slice of the AAV-Venus injected animals was proofed for fluorescence expression of Venus in area CA1 and CA3 (Zeiss, Axiovert 35). Slices absent of the fluorescence protein in the recording areas were excluded from further analysis.

Extracellular Field Recordings

Slices were placed in a submerged recording chamber and perfused with carbogenated ACSF (32° C.; 125 mM NaCl, 2 mM KCl, 1.25 mM NaH₂PO₄, 1 mM MgCl₂, 26 mM NaHCO₃, 2 mM CaCl₂, 25 mM glucose) at a rate of 1.2 to 1.5 ml/min. Field excitatory postsynaptic potentials (fEPSPs) were recorded in stratum radiatum of CA1 region with a borosilicate glass micropipette (resistance 2-4 MΩ) filled with 3 M NaCl at a depth of ˜150-200 μm. Monopolar tungsten electrodes were used for stimulating the Schaffer collaterals at a frequency of 0.1 Hz. Stimulation intensity was adjusted to 40% of maximum fEPSP slope for 20 minutes baseline recording. LTP was induced by applying theta-burst stimulation (TBS: 10 trains of 4 pulses at 100 Hz in an 200 ms interval, repeated 3 times). Basal synaptic transmission properties were analyzed via input-output-(IO) measurements and short-term plasticity was examined via paired pulse facilitation (PPF). The IO-measurements were performed either by application of a defined current values (25-175 μA) or by adjusting the stimulus intensity to certain fiber volley (FV) amplitudes (0.1-0.7 mV). PPF was performed by applying a pair of two closely spaced stimuli in different inter-stimulus-intervals (ISI) ranging from 10 to 160 ms.

Dendrite and Spine Analysis

Golgi-Cox Staining

Golgi staining was done using the FD Rapid GolgiStain™ Kit according to the manufacturer's instructions. All procedures were performed under dark conditions. One hemisphere of each mouse was used for electrophysiology and the other one for Golgi-Cox staining. Hemispheres were immersed in 2 ml mixtures of equal parts of kit solutions A and B and stored at RT for 2 weeks. Afterwards brain tissues were stored in solution C at 4° C. for at least 48 h and up to 7 days before sectioning. Solutions AB and C were renewed within the first 24 h. Coronal sections of 200 μm were cut with a vibrating microtome (Leica, VT1200S) while embedded in 2% Agar in 0.1 M PBS. Each section was mounted with Solution C on an adhesive microscope slide pre-coated with 1% gelatin/0.1% chromalaun on both sides and stained according to the manufacturer's protocol with the exception that AppliClear (AppliChem) was used instead of xylene. Finally slices were cover-slipped with Permount (Fisher Scientific).

Imaging and Analysis of Spine Density in Golgi-Cox Stained Slices

Imaging of 2′^{d} or 3^{rd} order dendritic branches of hippocampal pyramidal neurons of area CA3 and CA1 was done with an Axioplan 2 imaging microscope (Zeiss) using a 63× oil objective and a z-stack thickness of 0.5 μm under reflected light. The number of spines was determined per micrometer of dendritic length (in total 100 μm) at apical and basal compartments using ImageJ (1.48v, National Instruments of Health, USA). At minimum 4 animals per genotype and 4 neurons per animal were analyzed blinded to genotype and injected virus.

Data Analysis

Data of electrophysiological recordings were collected, stored and analyzed with LABVIEW software (National Instruments, Austin, Tex.). The initial slope of fEPSPs elicited by stimulation of the Schaffer collaterals was measured over time, normalized to baseline and plotted as average±SEM. Analysis of the PPF data was performed by calculating the ratio of the slope of the second fEPSP divided by the slope of the first one and multiplied by 100. Data of Golgi-Cox staining were analyzed using GraphPad Prism (Version, 5.01) software. Spine density is expressed as mean±SEM. Differences between genotypes were detected with one-way analysis of variance (ANOVA) followed by Bonferroni's post hoc test using IBM SPSS Statistics 21.

Results

AAV-APPsα Injection Mediates Efficient and Long Lasting Neuronal Expression of APPsα in the Hippocampus of APP/PS1ΔE9 Mice

To assess the therapeutic potential of APPsα we used AAV-mediated overexpression of APPsα in the brain of aged (12 month-old) APP/PS1ΔE9 mice. APP/PS1ΔE9 mice show progressive plaque deposition starting at about 5-6 months of age and highly abundant plaques are observed at 12 months of age (Jankowsky et al, 2004; Xiong et al, 2011). AAV9 vectors expressing either Venus or codon optimized HA-tagged murine APPsα (HA-APPsα) under the control of the neuronal synapsin promoter (further referred to as AAV-Venus and AAV-APPsα, data not shown) were bilaterally injected into the stratum lacunosum moleculare region of the dorsal hippocampus and into the dentate gyrus (data not shown) of 12 month-old male APP/PSAE9 mice.

To monitor vector-mediated Venus and APPsα expression, mice were sacrificed 5 months after injection. Immunohistochemistry using an HA-tag specific antibody revealed widespread expression of HA-APPsα not only in the hippocampus, but also in the cortical layers above the injected hippocampus (data not shown). Analysis of serial anteroposterior coronal sections demonstrated widespread HA-APPsα immunoreactivity (over 3.5 mm) in the hippocampus from −2.6 mm posterior to +0.9 mm anterior from the injection site (data not shown) and in the adjacent cortex. More detailed analysis showed prominent expression of vector-mediated HA-APPsα in the pyramidal cells of the subiculum, in the CA1, CA2 regions and in granular neurons of dentate gyrus (data not shown). Within the CA3 subfield HA-APPsα expression was detectable but considerably lower. As APPsα expression was driven by the neuron-specific synapsin promotor, HA-APPsα expression was restricted to neuronal cells as revealed by double immunostaining against NeuN (data not shown). Consistently, no expression was detectable in microglia (Iba1, data not shown) or in astrocytes (GFAP, data not shown). The AAV-Venus expression pattern was largely similar to that of AAV-APPsα.

Western blot analysis of hippocampal extracts confirmed vector-mediated HA-APPsα protein expression in all injected animals. Comparable levels of either HA-APPsα or Venus were detected in injected APP/PS1ΔE9 mice or nontransgenic littermates, respectively (data not shown). Altogether we demonstrate that our AAV based approach leads to efficient and long lasting APPsα expression in the hippocampus and adjacent cortex.

AAV-APPsα Treatment Rescues the Spatial Memory Impairment of APP/PS1ΔE9 Mice

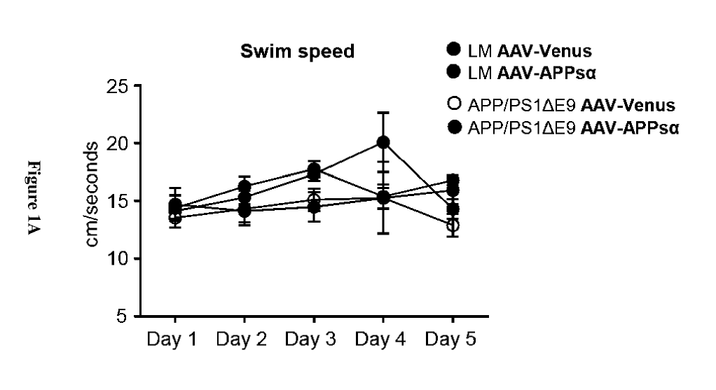

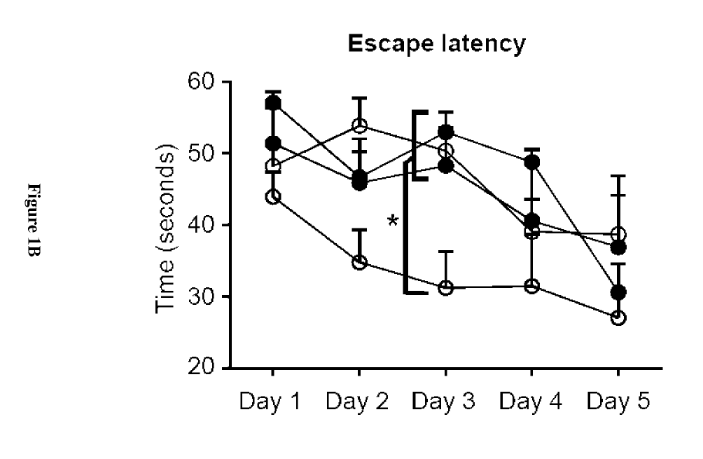

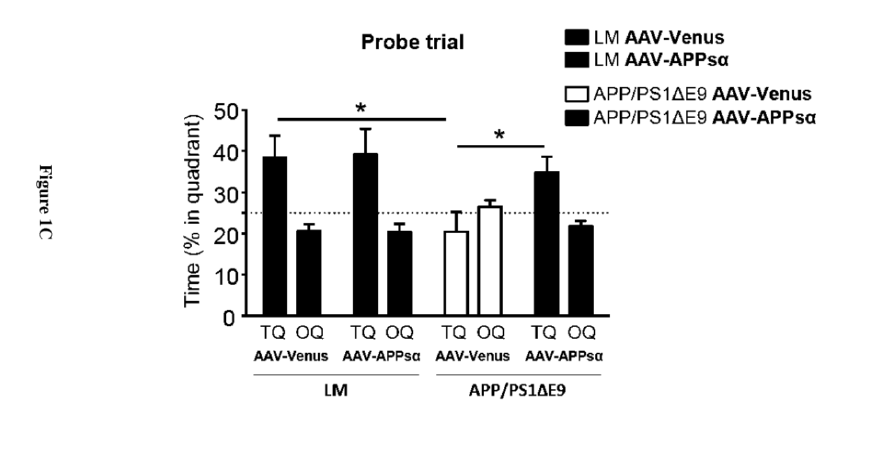

To analyze the consequences of AAV-APPsα or AAV-Venus injection for spatial learning and memory, mice were tested in the Morris water maze place navigation task (FIG. 1). To this end, transgenic APP/PS1ΔE9 mice (n=8 per group) or nontransgenic littermate controls (n=3-4 per group) were either injected with AAV-Venus or AAV-APPsα vectors at 12 months of age and tested 2 months later at 14 months of age. Swim speed was comparable in all groups of animals (FIG. 1A) over the 5 days of training, thus excluding impairments in motor performances. While all 4 groups of mice did show learning, as evidenced by reduced latency to reach the platform over the 5 days of training, we observed a group effect resulting from an overall significantly increased performance in nontransgenic littermates that had received AAV-APPsα (FIG. 1B). Injection of AAV-APPsα did not, however, improve the performance of APP/PS1ΔE9 mice (FIG. 1B). Similar results were obtained when analyzing the path length to reach the platform (data not shown). During the probe trial that assesses spatial reference memory and was conducted 72 hours after the last trial of training APP/PS1ΔE9 mice injected with AAV-Venus were strongly impaired (FIG. 1C) in comparison to littermate mice injected with AAV-Venus and showed no preference for the trained target quadrant (FIG. 1C; paired t-test: t₇=0.96; p=0.37). Strikingly, APP/PS1ΔE9 mice that had been injected with AAV-APPsα showed a clear preference for the trained target quadrant (FIG. 1C; paired t-test: t₇=2.516; p=0.045), that was statistically indistinguishable from the performance of littermate controls (FIG. 1C; p>0.84, 2-way ANOVA followed by Tukey's post-hoc test). Thus, vector mediated APPsα expression rescued the spatial memory impairment in aged APP/PS1ΔE9 mice despite established plaque deposition.

Impaired Synaptic Plasticity and Reduced Spine Density of APP/PS1ΔE9 Mice are Rescued by AAV-APPsα Expression

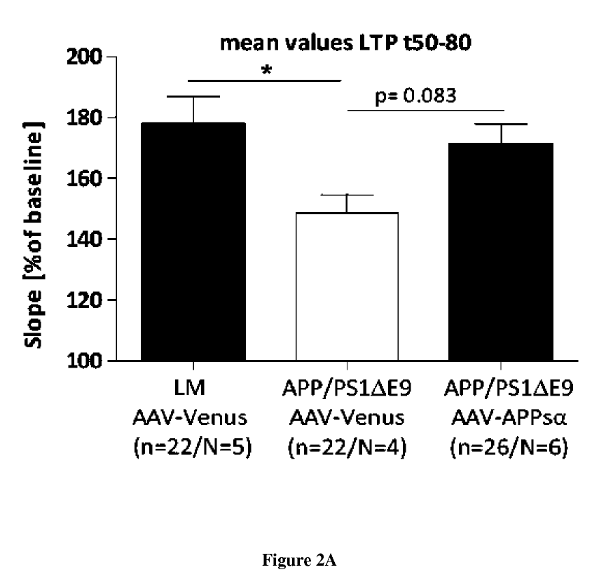

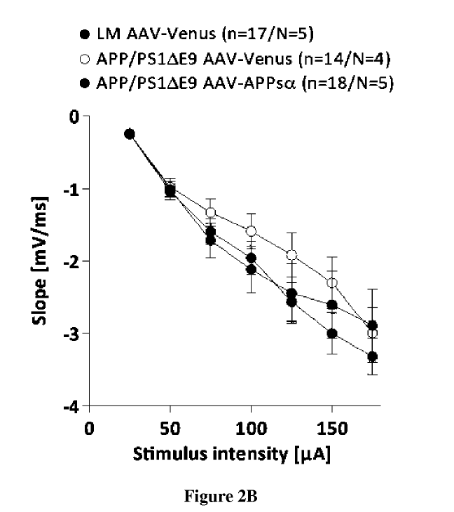

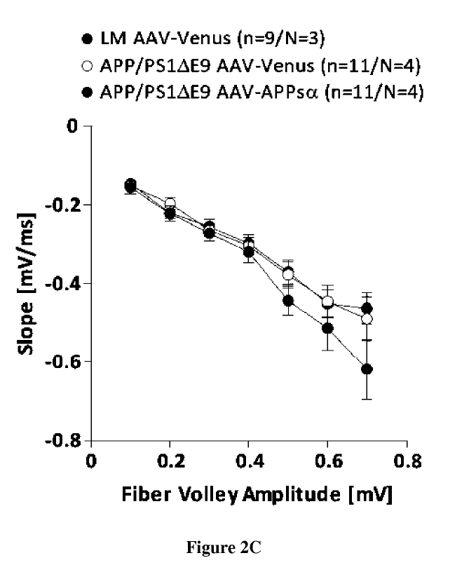

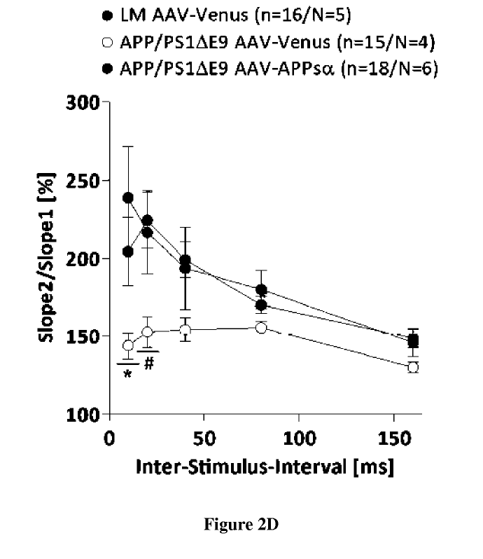

Having established that AAV-APPsα expression restored the spatial memory deficits of APP/PS1ΔE9 mice we evaluated whether these improvements were also reflected at the functional neuronal network level. We analyzed synaptic plasticity which is considered to represent the basis of newly formed declarative memory, 4-5 months after AAV injection at an age of 12-13 months. To this end, we induced long term potentiation (LTP) at the Schaffer collateral to CA1 pathway by theta-burst stimulation (TBS) after baseline recording (data not shown). Consistent with our previous results in noninjected APP/PS1ΔE9 mice (Heneka et al, 2013) AAV-Venus injected APP/PS1ΔE9 mice exhibited significantly lower induction and maintenance of LTP (n=22 slices), as compared to AAV-Venus injected littermate controls (n=22, data not shown). Nontransgenic control slices showed at the stable phase of LTP (t50-80 min after TBS) a potentiation of 178.01±8.98%, that was significantly reduced to only 148.47±6.04% in AAV-Venus injected APP/PS1ΔE9 mice (FIG. 2A; p=0.021, 1-way ANOVA followed by Bonferroni's post-hoc test). In contrast, the LTP curve recorded from AAV-APPsα injected APP/PS1ΔE9 slices (n=26) closely overlapped with and was statistically indistinguishable (1-Way ANOVA for t50-80, p>1) from that of nontransgenic littermate controls (data not shown). AAV mediated expression of APPsα largely ameliorated LTP deficits of APP/PS1ΔE9 mice as evidenced by nearly identical average potentiation at t50-80 in AAV-APPsα treated APP/PS1ΔE9 mice (171.48±6.29%) and littermate controls (178.01±8.98%) receiving AAV-Venus control virus (FIG. 2A). While basal synaptic transmission was comparable in all groups (FIGS. 2B and C), short-term synaptic plasticity evaluated by paired pulse facilitation (PPF, FIG. 2D) was significantly impaired in APP/PS1ΔE9 mice. Transgenic animals injected with AAV-Venus showed an overall lowered response towards the second stimulus in the PPF paradigm, reaching significance at an inter-stimulus interval (ISI) of 10 ms compared to littermate controls (p=0.03; 1-way ANOVA followed by Bonferroni's post-hoc test). Strikingly, AAV-APPsα treatment completely rescued presynaptic functionality in APP/PS1ΔE9 animals, as evidenced by PPF values statistically indistinguishable from littermate controls and significantly different from that of AAV-Venus injected transgenic animals (p(ISI₂₀ₘₛ)=0.047; FIG. 2D).

Next we evaluated spine density as a correlate of excitatory synapses in the same set of animals as used for electrophysiology. Previous studies had indicated reduced spine density in various AD mouse models, presumably due to Aβ mediated toxic effects (reviewed in (Spires-Jones & Knafo, 2012). Spine density of basal and mid-apical dendritic segments of hippocampal CA1 and CA3 pyramidal cells was assessed using Golgi staining (data not shown). Apical dendrites of CA1 neurons showed comparable spine density between experimental groups, whereas significantly reduced spine density was observed in the basal dendrites of CA1 neurons from APP/PS1ΔE9 mice (n=16 neurons) as compared to littermates controls (n=24 neurons, both treated with AAV-Venus, FIG. 2E). Analysis of CA3 neurons revealed significantly fewer spines in both basal (t-test, p=0.01) and apical (p=0.014) dendritic segments when comparing AAV-Venus expressing APP/PS1ΔE9 mice and nontransgenic littermates controls. Importantly, AAV-APPsα overexpression partially restored spine density in CA3 apical segments (n=24) and completely rescued the spine density deficit in basal dendrites of CA3 neurons from APP/PS1ΔE9 mice (p=0.031; FIG. 2F). Together, these data indicate that APPsα expression substantially ameliorates both structural and functional synaptic impairments of aged AD model mice.

AAV-APPsα Expression Decreases Aβ Levels and Plaque Deposition in Aged APP/PS1dE9 Mice

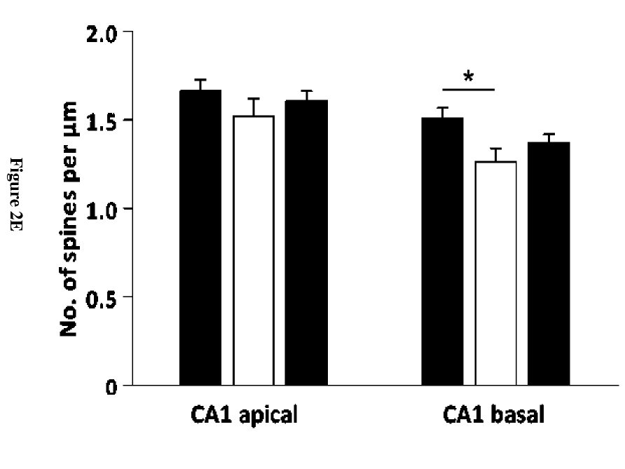

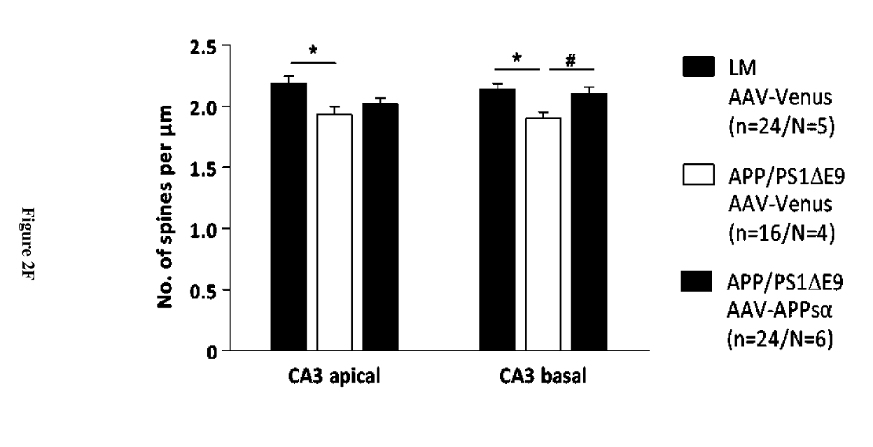

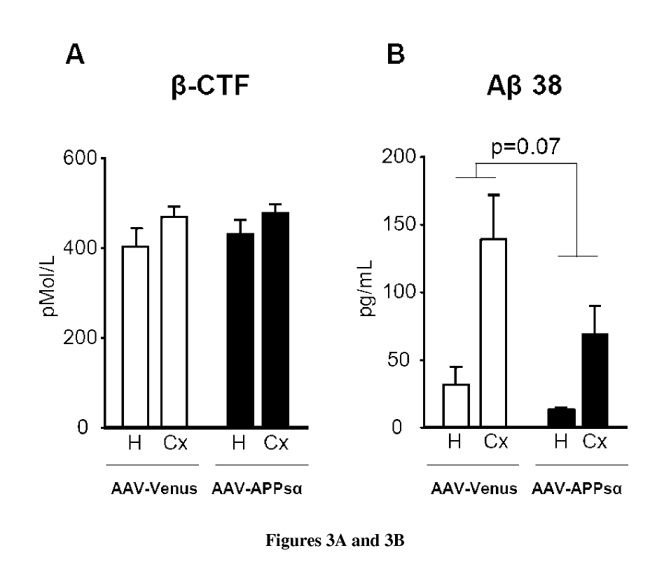

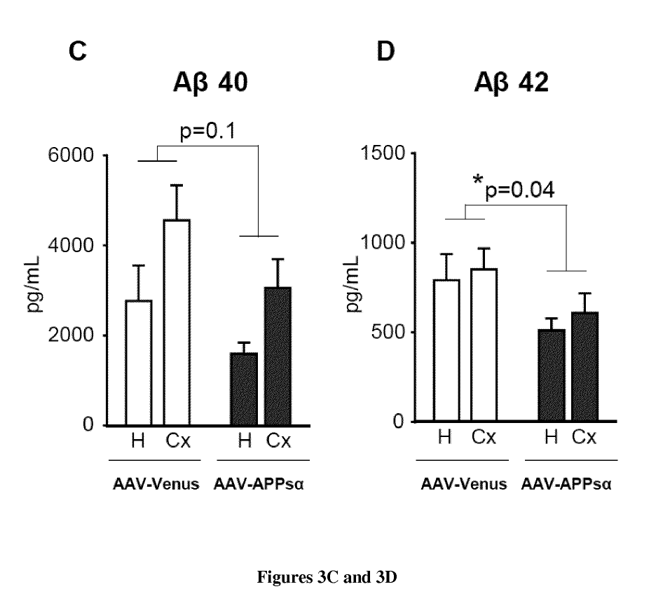

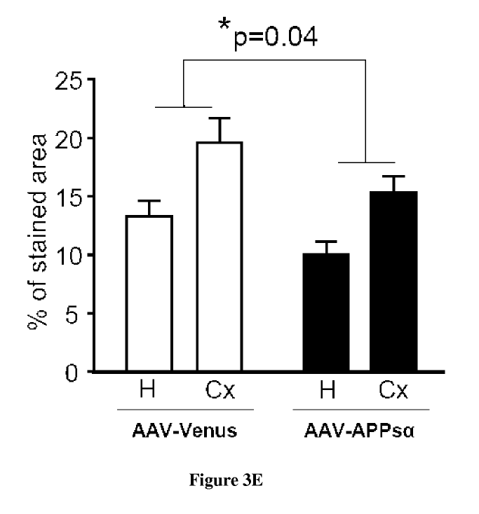

APPsα had previously been reported to bind to BACE-1 and thereby reduce Aβ production (Obregon et al, 2012). We therefore evaluated if beneficial effects of AAV-APPsα overexpression on synaptic plasticity and cognitive function were associated with reduced amyloidogenic processing of APP. Employing a sensitive electrochemiluminescence ELISA we quantified products of amyloidogenic metabolism (Aβ and β-CTF) in the cortex (Cx) and hippocampus (H) of 17 months old APP/PS1ΔE9 mice (n=8/group), 5 months after viral vector injection. No significant difference in β-CTF levels were detectable in APP/PS1ΔE9 mice injected with AAV-APPsα vector, as compared to mice injected with AAV-Venus control vector (FIG. 3A). In contrast, we observed a significant decrease in soluble Aβ42 (reduced by about 33% vs control, FIG. 3D) in both cortex and hippocampus of APP/PS1ΔE9 mice injected with AAV-APPsα vector, as compared to AAV-Venus control injections. Similarly, we found a trend towards decreased amounts of Aβ38 and Aβ40 that did, however, not reach statistical significance (FIG. 3B, C).

In order to assess the impact of APPsα overexpression on amyloid deposition, we used 4G8 immunostaining to quantify the area covered by plaques both in the hippocampus and cortex of 17 months old APP/PS1ΔE9 mice injected with viral vectors (data not shown). Interestingly, AAV-APPsα injection (n=8) resulted in a significantly reduced plaque area both in cortex and hippocampus as compared to AAV-Venus injected controls (FIG. 3E). Together, these results indicate that AAV-mediated APPsα overexpression moderately reduces both Aβ generation and amyloid plaque load in APP/PS1ΔE9 mice not only in the AAV injected hippocampus but also in distant cortical areas.

AAV-APPsα Induces Microglia Recruitment and Activation in the Vicinity of Amyloid Plaques

Accumulation of amyloid plaques in APP/PS1ΔE9 mice has previously been shown to be accompanied by microgliosis and astrocytosis notably at advanced stages of plaque pathology (Kamphuis et al, 2012; Prokop et al, 2013). Here we evaluated the expression of GFAP (as an astrocyte-specific marker) and Iba1 (as a microglial marker) by Western blot analysis (FIG. 4A) and IHC (FIG. 4B) in the hippocampus of 17 month old APP/PS1ΔE9 mice treated either with AAV-APPsα or control vector. While no significant difference was detectable for the astroglial marker GFAP, AAV-APPsα treatment lead to a significant increase in Iba1 expression (about +44%; t-test, p=0.003; FIG. 4A), as compared to AAV-Venus control injections. Staining of brain sections further confirmed these data (data not shown) at the cellular level. We went on and quantified GFAP and Iba1 immunoreactivity around amyloid plaques in the hippocampus. Consistent with Western blot analysis, GFAP immunoreactivity was not affected by AAV-APPsα injection (FIG. 4B). In contrast, the reduction of amyloid deposits observed after injection of the AAV-APPsα vector in APP/PS1ΔE9 mice was accompanied by a 2.3-fold increase in Iba1 immunoreactivity in the vicinity of plaques (FIG. 4B). Moreover, we observed an altered morphology of microglia in AAV-APPsα treated mice characterized by increased ramifications in AAV-APPsα versus control vector injected APP/PS1ΔE9 mice (data not shown). Microglia contribute to Aβ clearance and are thought to play a protective role at least during early stages of AD (Prokop et al, 2013). Indeed, plaque associated microglia (from both AAV-APPsα and AAV-Venus treated mice) were also engaged in Aβ uptake as evidenced by Iba1/4G8 double staining (data not shown). Recently, genetic variants of TREM2 (Triggering Receptor Expressed on Myeloid cells) have been associated with an increased risk for AD (Guerreiro et al, 2013; Jonsson et al, 2013). Although the precise role of TREM2 for AD pathogenesis and Aβ pathology is still controversial (Jay et al, 2015; Wang et al, 2015) TREM2 expression has been consistently detected in plaque associated Iba1⁺ cells in AD model mice (Frank et al, 2008; Jay et al, 2015). Consistent with an increase in plaque associated microglia we detected a significant increase of TREM2 expression (about 60% of control, t-test, p<0.05) by Western blot analysis in hippocampi of APP/PS1ΔE9 mice injected with AAV-APPsα versus controls (FIG. 4C, n=8 per group). We also determined the expression of neprilysin (NEP) and insulin-degrading enzyme (IDE) that are proteases produced by microglia that contribute to Aβ clearance (Tang 2008). Expression of NEP was identical in APP/PS1ΔE9 mice injected with AAV-APPsα versus control (not shown). However a significant increase (of about +20%, t-test, p<0.001) in IDE expression was observed after AAV-HA-APPsα vector injection (FIG. 4C). Together these data suggest that AAV mediated APPsα expression induces microglia recruitment, activation and possibly also phagocytic function which may lead to enhanced Aβ and plaque clearance.

AAV-APPsβ Injection Induce an Efficient and Durable Expression of Hippocampal Neurons in APP/PS1ΔE9 Mice

In order to assess the consequences of APPsβ neuronal overexpression in APP/PS1ΔE9 mice, AAV9-Venus or AAV9-APPsβ and AAV9-APPsα both hemaglutinine tagged (thereafter referred as AAV-Venus, AAV-APPsβ and AAV-APPsα) (data not shown) were bilaterally injected into the stratum lacunosum moleculare and the dentate gyrus regions of the hippocampus of aged APP/PS1ΔE9 mice (12 months) (data not shown). Mice were sacrificed at 17 months of age, (5 months post-injection) to evaluate the expression of both APPsα and APPsβ. Efficient transduction of the hippocampus (especially in the CA1, CA2 and dentate gyrus) was evidenced. The pattern of expression in the hippocampus was similar in AAV-APPsα injected mice and diffusion of APPsβ expression into the peri-hippocampal cortex was observed (data not shown). The APPsβ expression showed also a nice diffusion from rostral to caudal coordinates in both cortex and hippocampus (data not shown). Further cellular analysis of the CA1 layer confirmed that the synapsin promoter allowed specific and efficient neuronal transduction without any transduction of neither astrocytes nor microglia (data not shown). Levels of expression of APPsβ-HA and APPsα-HA in hippocampus analyzed by western blot were very close and consistent in every single animal injected. AAV-Venus control animals also displayed a similar level of Venus (data not shown).

AAV-APPsβ does not Improve Spatial Reference Memory of Aged APP/PS1ΔE9

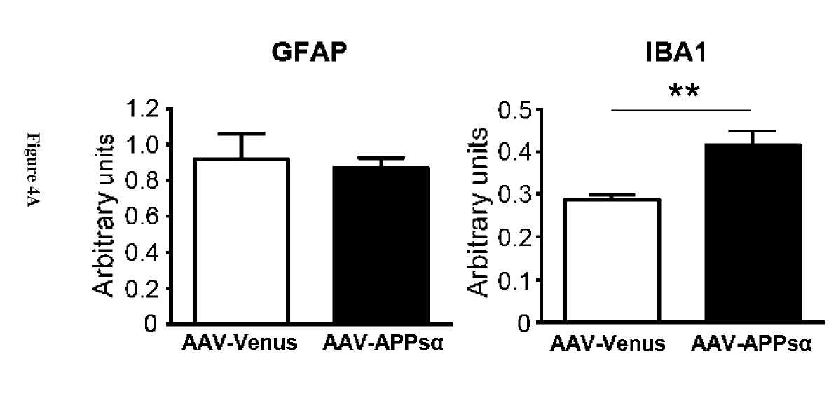

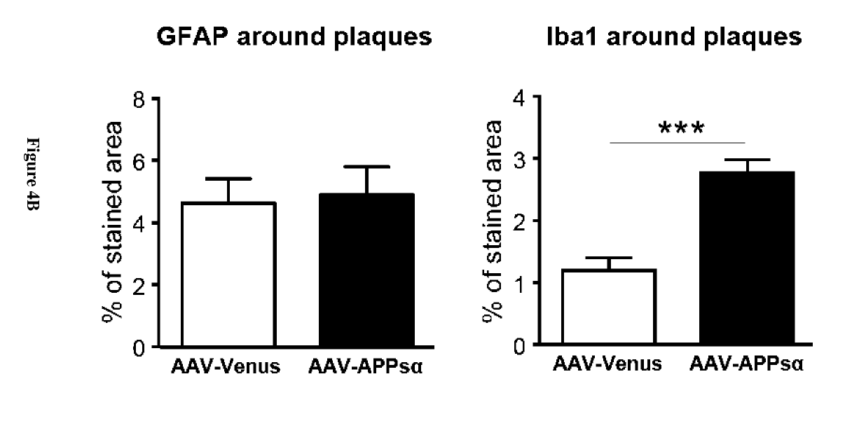

We used the Morris water maze to evaluate the spatial reference of the APP/PS1ΔE9 mice. Transgenic mice (AAV-Venus (n=8), AAV-APPsα (n=8) or AAV-APPsβ (n=7)) or littermate controls (AAV-Venus (n=3)) were injected at 12 months old and assessed 2 months later at 14 months of age. During the five days of the training phase (TQ, data not shown), every group of animals showed an efficient learning of the platform position highlighted by a decreased distance to find it over the five days (data not shown). 72 hours after the last training session, the platform was removed in order to assess spatial reference memory evidenced by the distance spent in the TQ (FIG. 5). APP/PS1ΔE9 control mice injected with AAV-Venus showed an impaired memory as compared to AAV-Venus injected littermate mice (Tukey post-hoc test: p=0.04). AAV-APPsα injection in transgenic mice improved spatial reference memory as previously shown (p=0.01). In contrast, however, AAV-APPsβ treatment did rescue spatial reference memory as the time spent in the TQ was equivalent to AAV-Venus mice (p=0.99) and significantly different compared to AAV-APPsα mice (p=0.02). The lack of efficiency of AAV-APPsβ to restore an efficient search strategy is highlighted by the occupancy plots (data not shown). Together, these results demonstrate that, in contrast to AAV-APPsα, AAV-APPβ injection is not able to restore memory deficits of APP/PS1ΔE9.

AAV-APPsβ Injection does not Restore Long-Term Potentiation in the Hippocampus of Aged APP/PS1ΔE9 Mice.

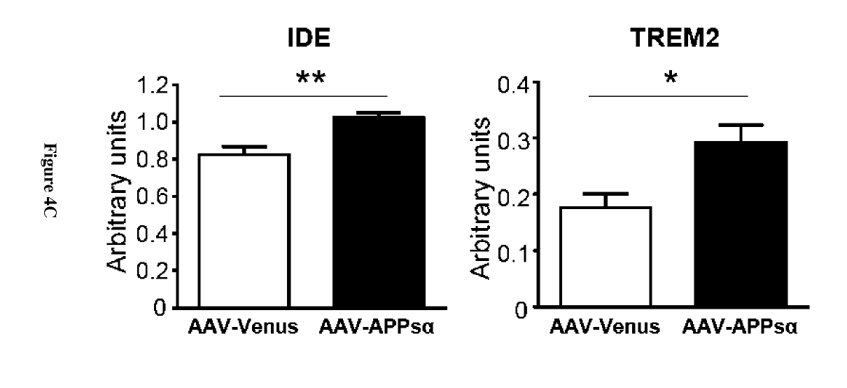

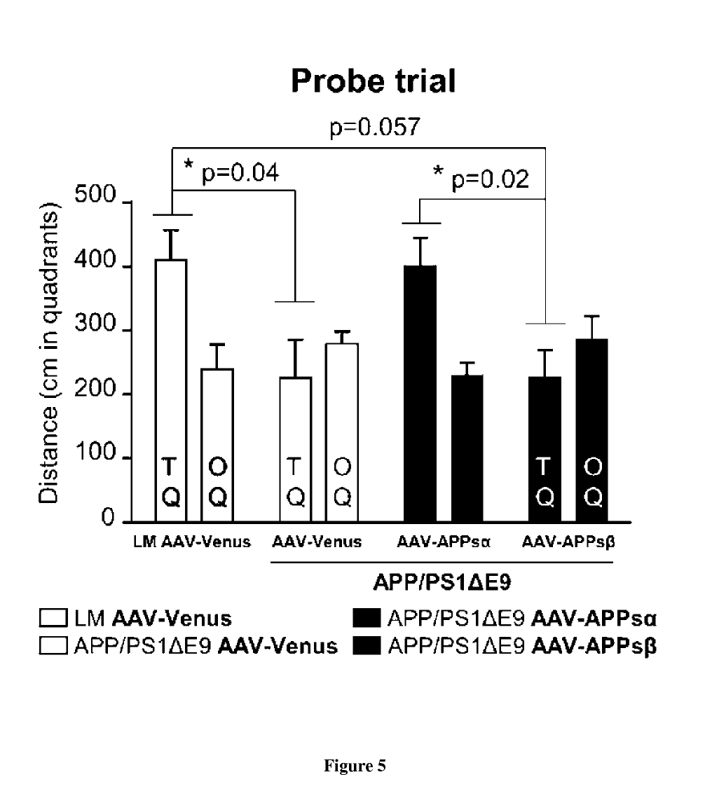

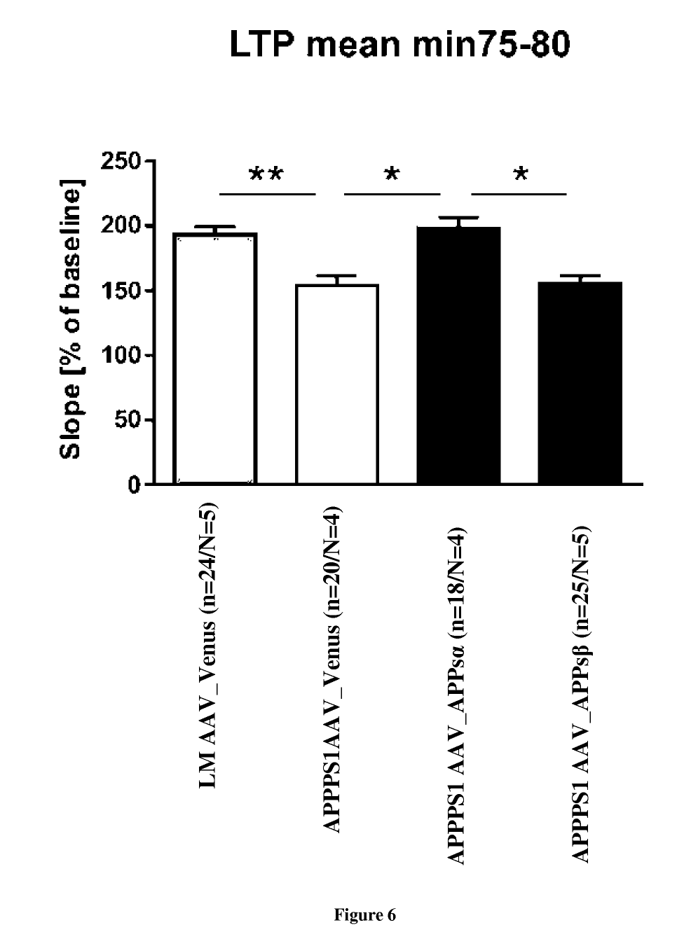

We then evaluated whether the improvements of spatial memory deficits in APP/PS1ΔE9 mice were also reflected at the functional neuronal network level. Up to know the effects of APPsβ on synaptic plasticity had not been studied, neither in vitro nor in vivo. To analyze synaptic plasticity we induced long-term potentiation (LTP) at the Schaffer collateral to CA1 pathway by theta-burst stimulation (TBS) after baseline recording (FIG. 6). Acute slices of AAV-Venus injected APP/PS1ΔE9 animals exhibited significant lower induction and maintenance of LTP compared to littermate controls indicating a significant impairment of the transgenic mice. Viral expression of APPsα restored potentiation after TBS in transgenic animals and resulted in an LTP curve progression comparable to that of LM controls, confirming previous results (FIG. 2). Injection of AAV-APPsβ vector in APP/PS1ΔE9 mice did also not correct the impairment of the presynaptic compartment as shown by altered PPF at the 10 ms ISI, This parameter was restored after AAV-APPsα injection but not with AAV-APPsβ (data not shown). Together, these data indicate that APPsβ expression in contrast to APPsa expression fails to ameliorate functional synaptic impairments of aged AD model mice. This in turn indicates a crucial role for the last 16 aminoacids of APPsa (that are lacking in APPsb) as a domain that mediates the rescue effect on memory and synaptic plasticity.

AAV-APPsβ does not Activate Microglia In Vivo in Aged APP/PS1ΔE9 Mice

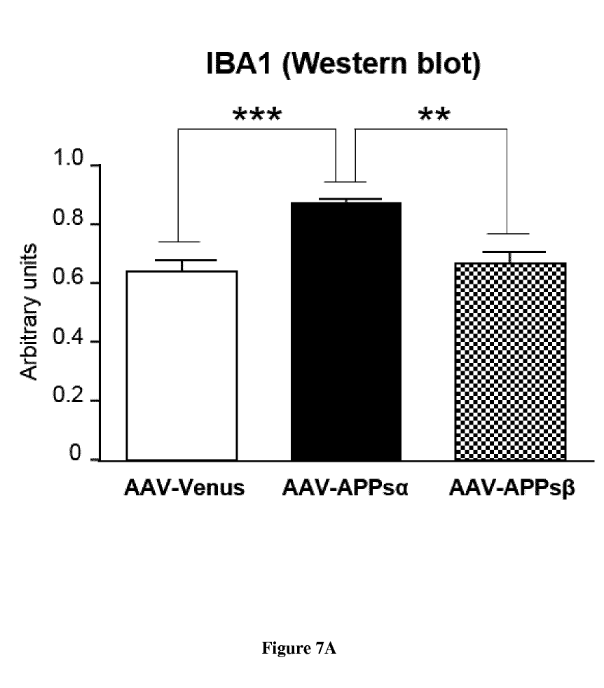

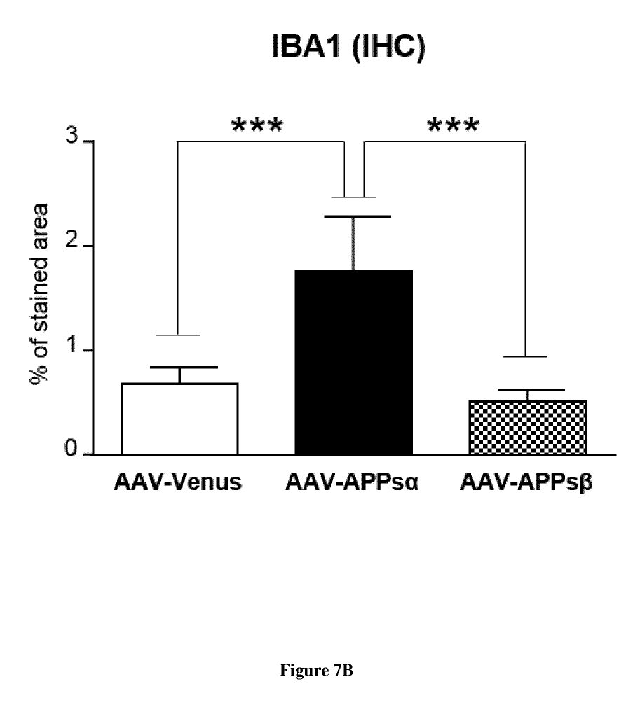

In order to explain the discrepancy between AAV-APPsα and AAV-APPsβ regarding amyloid plaques degradation, we assessed microglial activation. We first confirmed that APPsα is able activate microglia which in turn internalize Aβ and upregulate the amyloid degrading enzyme IDE and the receptor TREM2. In sharp contrast, the activation of microglia evidenced by upregulation of Iba1 in both western blot and immunohistochemistry was not observed after AAV-APPsβ or AAV-Venus injection (FIG. 7). This result is unexpected and novel as previous in vitro studies indicated that both APPsα and APPsβ may activate microglia in culture.

This results might indicate that the AAV-APPsα effects on soluble Aβ one hand and amyloid plaques the other hand are mediated by two independent mechanisms. Finally, the lack of activation following APPsβ injection could explain why plaques levels are not altered.

### CONCLUSION

Despite a recent shift of research efforts towards preventive strategies, there is still an urgent lack of an effective treatment of patients with clinically established AD. So far, many therapeutic approaches targeted the secretases processing APP. However, since all secretases act on many different substrates besides APP (Prox et al, 2012; Vassar et al, 2014), these strategies have major drawbacks for clinical application. γ-secretase is physiologically essential and current clinical trials to develop γ-secretase inhibitors have been abrogated due to serious side effects, likely resulting from impaired Notch signaling (Doody et al, 2013). Also systemic upregulation of the major α-secretase ADAM10 to boost APPsα production is problematic, as this may enhance cleavage of substrates implicated in tumorigenesis and in addition of several hundred substrates expressed in neurons (reviewed by Nhan et al, 2015; Prox et al, 2012, Kuhn et al., 2016). Thus, direct overexpression of APPsα in the brain may be more promising than pharmacological upregulation of α-secretase.

Here, the inventors explored a gene therapeutic approach and used AAV-based gene transfer to overexpress APPsα in the brain of transgenic APP/PS1ΔE9 mice that have been widely used in experimental studies assessing the efficacy of AD therapies. Bigenic APP_{SWE}/PS1ΔE9 mice express a chimeric mouse/human APP (with Swedish double mutation) and a mutant human PS1 gene (PS1ΔE9) both associated with familial forms of AD. They produce high amounts of huAβ leading to amyloid deposition starting at 5-6 months and pronounced progression of plaque pathology with age that is associated with impairments in cognitive behavior (Savonenko et al, 2005). Using bilateral injection of AAV-APPsα vector particles the inventors achieved highly efficient and widespread expression of APPsα throughout the whole hippocamus and also in adjacent cortical areas.

In this study, a single bilateral injection of AAV-APPsα particles was sufficient to mediate long-lasting APPsα expression over five months that was well tolerated without apparent adverse effects. This was a crucial prerequisite to study potential therapeutic efficacy of AAV-APPsα overexpression. To this end, they used aged (12 month old) APP/PS1ΔE9 mice with preexisting amyloidosis to mimic the situation in AD patients that are usually clinically diagnosed many years after the onset of pathology (Villemagne et al, 2013).

Taken together, the inventors provide evidence that APPsα as a molecule has beneficial effects on cognition, synaptic density, synaptic function and plasticity, microglia activation and reduces both soluble Aβ and insoluble Aβ deposits in the form of plaques.

Moreover, they show that APPsα and not APPsβ is responsible for the positive rescue effects in an Alzheimer mouse model. They show that APPsα but not APPsβ rescues: a) cognition (MWM) and b) synaptic plasticity as a molecular correlate to synaptic strength. This are totally novel data not reported so far. In addition they also show that APPsα activates microglia and recruits microglia towards plaques. This is an unexpected novel in vivo finding, as previously in vitro both APPsα and APPsβ could activate microglia to secrete inflammatory cytokines. The inventors show for the first time that in vivo both molecules have different properties and that APPsα could be very useful in AD and Down syndrome treatment strategy.

### REFERENCES

Throughout this application, various references, including United States patents and patent applications, describe the state of the art to which this invention pertains. The disclosures of these references are hereby incorporated by reference in entirety into the present disclosure.
Ahmed R R, Holler C J, Webb R L, Li F, Beckett T L, Murphy M P (2010) BACE1 and BACE2 enzymatic activities in Alzheimer's disease. J Neurochem 112: 1045-1053
Austin S A, Combs C K (2008) Mechanisms of Microglial Activation by Amyloid precursor Protein and its Proteolytic Fragments. In Central Nervous System Diseases and Inflammation, Lane T E, Carson M, Bergmann C, Wyss-Coray T (eds) pp 13-32. Springer US
Aydin D, Weyer S W, Muller U C (2012) Functions of the APP gene family in the nervous system: insights from mouse models. Exp Brain Res 217: 423-434
Bell K F, Zheng L, Fahrenholz F, Cuello A C (2008) ADAM-10 over-expression increases cortical synaptogenesis. Neurobiol Aging 29: 554-565
Berger A, Lorain S, Josephine C, Desrosiers M, Peccate C, Voit T, Garcia L, Sahel J A, Bemelmans A P (2015) Repair of Rhodopsin mRNA by Spliceosome-Mediated RNA Trans-Splicing: A New Approach for Autosomal Dominant Retinitis Pigmentosa. Mol Ther
Bodles A M, Barger S W (2005) Secreted beta-amyloid precursor protein activates microglia via JNK and p38-MAPK. Neurobiol Aging 26: 9-16
Borchelt D R, Ratovitski T, van Lare J, Lee M K, Gonzales V, Jenkins N A, Copeland N G, Price D L, Sisodia S S (1997) Accelerated amyloid deposition in the brains of transgenic mice coexpressing mutant presenilin 1 and amyloid precursor proteins. Neuron 19: 939-945
Bour A, Little S, Dodart J C, Kelche C, Mathis C (2004) A secreted form of the beta-amyloid precursor protein (sAPP695) improves spatial recognition memory in OF1 mice. Neurobiol Learn Mem 81: 27-38
Caille I, Allinquant B, Dupont E, Bouillot C, Langer A, Muller U, Prochiantz A (2004) Soluble form of amyloid precursor protein regulates proliferation of progenitors in the adult subventricular zone. Development 131: 2173-2181
Casanova F, Carney P R, Sarntinoranont M (2014) Effect of needle insertion speed on tissue injury, stress, and backflow distribution for convection-enhanced delivery in the rat brain. PloS one 9: e94919
Claasen A M, Guevremont D, Mason-Parker S E, Bourne K, Tate W P, Abraham W C, Williams J M (2009) Secreted amyloid precursor protein-alpha upregulates synaptic protein synthesis by a protein kinase G-dependent mechanism. Neurosci Lett 460: 92-96
Corrigan F, Vink R, Blumbergs P C, Masters C L, Cappai R, van den Heuvel C (2012) sAPPalpha rescues deficits in amyloid precursor protein knockout mice following focal traumatic brain injury. J Neurochem 122: 208-220
Cousins S L, Hoey S E, Anne Stephenson F, Perkinton M S (2009) Amyloid precursor protein 695 associates with assembled NR2A- and NR2B-containing NMDA receptors to result in the enhancement of their cell surface delivery. J Neurochem 111: 1501-1513
Cousins S L, Innocent N, Stephenson F A (2013) Neto1 associates with the NMDA receptor/amyloid precursor protein complex. J Neurochem 126: 554-564
Dobrowolska J A, Kasten T, Huang Y, Benzinger T L, Sigurdson W, Ovod V, Morris J C, Bateman R J (2014) Diurnal patterns of soluble amyloid precursor protein metabolites in the human central nervous system. PloS one 9: e89998
Doody R S, Raman R, Farlow M, Iwatsubo T, Vellas B, Joffe S, Kieburtz K, He F, Sun X, Thomas R G et al (2013) A phase 3 trial of semagacestat for treatment of Alzheimer's disease. N Engl J Med 369: 341-350
Endres K, Fahrenholz F (2012) Regulation of alpha-secretase ADAM10 expression and activity. Exp Brain Res 217: 343-352
Frank S, Burbach G J, Bonin M, Walter M, Streit W, Bechmann I, Deller T (2008) TREM2 is upregulated in amyloid plaque-associated microglia in aged APP23 transgenic mice. Glia 56: 1438-1447
Furukawa K, Mattson M P (1998) Secreted amyloid precursor protein alpha selectively suppresses N-methyl-D-aspartate currents in hippocampal neurons: involvement of cyclic GMP. Neuroscience 83: 429-438
Furukawa K, Sopher B L, Rydel R E, Begley J G, Pham D G, Martin G M, Fox M, Mattson M P (1996) Increased activity-regulating and neuroprotective efficacy of alpha-secretase-derived secreted amyloid precursor protein conferred by a C-terminal heparin-binding domain. J Neurochem 67: 1882-1896
Goodman Y, Mattson M P (1994) Secreted forms of beta-amyloid precursor protein protect hippocampal neurons against amyloid beta-peptide-induced oxidative injury. Exp Neurol 128: 1-12
Guerreiro R, Wojtas A, Bras J, Carrasquillo M, Rogaeva E, Majounie E, Cruchaga C, Sassi C, Kauwe J S, Younkin S et al (2013) TREM2 variants in Alzheimer's disease. N Engl J Med 368: 117-127
Hardy, J. and D. Allsop (1991). “Amyloid deposition as the central event in the aetiology of Alzheimer's disease.” Trends Pharmacol Sci 12(10): 383-388.
Heneka M T, Kummer M P, Stutz A, Delekate A, Schwartz S, Vieira-Saecker A, Griep A, Axt D, Remus A, Tzeng T C et al (2013) NLRP3 is activated in Alzheimer's disease and contributes to pathology in APP/PS1 mice. Nature 493: 674-678
Hick M, Herrmann U, Weyer S W, Mallm J P, Tschape J A, Borgers M, Mercken M, Roth F C, Draguhn A, Slomianka L et al (2015) Acute function of secreted amyloid precursor protein fragment APPsalpha in synaptic plasticity. Acta Neuropathol 129: 21-37
Hick, M., U. Herrmann, S. W. Weyer, J. P. Mallm, J. A. Tschape, M. Borgers, M. Mercken, F. C. Roth, A. Draguhn, L. Slomianka, D. P. Wolfer, M. Korte and U. C. Muller (2015). “Acute function of secreted amyloid precursor protein fragment APPsalpha in synaptic plasticity.” Acta Neuropathol 129(1): 21-37.
Hoe H S, Lee H K, Pak D T (2012) The upside of APP at synapses. CNS Neurosci Ther 18: 47-56
Hoey S E, Williams R J, Perkinton M S (2009) Synaptic NMDA receptor activation stimulates alpha-secretase amyloid precursor protein processing and inhibits amyloid-beta production. J Neurosci 29: 4442-4460
Holsinger R M, McLean C A, Beyreuther K, Masters C L, Evin G (2002) Increased expression of the amyloid precursor beta-secretase in Alzheimer's disease. Annals of neurology 51: 783-786
Ishida A, Furukawa K, Keller J N, Mattson M P (1997) Secreted form of beta-amyloid precursor protein shifts the frequency dependency for induction of LTD, and enhances LTP in hippocampal slices. Neuroreport 8: 2133-2137
Jankowsky J L, Fadale D J, Anderson J, Xu G M, Gonzales V, Jenkins N A, Copeland N G, Lee M K, Younkin L H, Wagner S L et al (2004) Mutant presenilins specifically elevate the levels of the 42 residue beta-amyloid peptide in vivo: evidence for augmentation of a 42-specific gamma secretase. Hum Mol Genet 13: 159-170
Jay T R, Miller C M, Cheng P J, Graham L C, Bemiller S, Broihier M L, Xu G, Margevicius D, Karlo J C, Sousa G L et al (2015) TREM2 deficiency eliminates TREM2+ inflammatory macrophages and ameliorates pathology in Alzheimer's disease mouse models. J Exp Med 212: 287-295
Jonsson T, Stefansson H, Steinberg S, Jonsdottir I, Jonsson P V, Snaedal J, Bjornsson S, Huttenlocher J, Levey A I, Lah J J et al (2013) Variant of TREM2 associated with the risk of Alzheimer's disease. N Engl J Med 368: 107-116
Jonsson, T., J. K. Atwal, S. Steinberg, J. Snaedal, P. V. Jonsson, S. Bjornsson, H. Stefansson, P. Sulem, D. Gudbjartsson, J. Maloney, K. Hoyte, A. Gustafson, Y. Liu, Y. Lu, T. Bhangale, R. R. Graham, J. Huttenlocher, G. Bjornsdottir, O. A. Andreassen, E. G. Jonsson, A. Palotie, T. W. Behrens, 0. T. Magnusson, A. Kong, U. Thorsteinsdottir, R. J. Watts and K. Stefansson (2012). “A mutation in APP protects against Alzheimer's disease and age-related cognitive decline.” Nature 488(7409): 96-99.
Kamphuis W, Mamber C, Moeton M, Kooijman L, Sluijs J A, Jansen A H, Verveer M, de Groot L R, Smith V D, Rangarajan S et al (2012) GFAP isoforms in adult mouse brain with a focus on neurogenic astrocytes and reactive astrogliosis in mouse models of Alzheimer's disease. PloS one 7: e42823
Klevanski M, Saar M, Baumkotter F, Weyer S W, Kins S, Muller U C (2014) Differential role of APP and APLPs for neuromuscular synaptic morphology and function. Mol Cell Neurosci 61C: 201-210
Kogel D, Deller T, Behl C (2012) Roles of amyloid precursor protein family members in neuroprotection, stress signaling and aging. Exp Brain Res 217: 471-479
Kuhn P H, Colombo A V, Schusser B, Dreymueller D, Wetzel S, Schepers U, Herber J, Ludwig A, Kremmer E, Montag D, Müller U, Schweizer M, Saftig P, Brase S, Lichtenthaler S F (2016) Systematic substrate identification indicates a central role for the metalloprotease ADAM10 in axon targeting and synapse function. eLife: 2016; available online DOI:10.7554/eLife.12748
Lannfelt L, Basun H, Wahlund L O, Rowe B A, Wagner S L (1995) Decreased alpha-secretase-cleaved amyloid precursor protein as a diagnostic marker for Alzheimer's disease. Nat Med 1: 829-832
Lassek M, Weingarten J, Einsfelder U, Brendel P, Muller U, Volknandt W (2013) Amyloid precursor proteins are constituents of the presynaptic active zone. J Neurochem 127: 48-56
Lee K J, Moussa C E, Lee Y, Sung Y, Howell B W, Turner R S, Pak D T, Hoe H S (2010) Beta amyloid-independent role of amyloid precursor protein in generation and maintenance of dendritic spines. Neuroscience 169: 344-356
Leissring M A, Farris W, Chang A Y, Walsh D M, Wu X, Sun X, Frosch M P, Selkoe D J (2003) Enhanced proteolysis of beta-amyloid in APP transgenic mice prevents plaque formation, secondary pathology, and premature death. Neuron 40: 1087-1093
Leyssen M, Ayaz D, Hebert S S, Reeve S, De Strooper B, Hassan B A (2005) Amyloid precursor protein promotes post-developmental neurite arborization in the Drosophila brain. EMBO J 24: 2944-2955
Lichtenthaler S F, Haass C, Steiner H (2011) Regulated intramembrane proteolysis—lessons from amyloid precursor protein processing. J Neurochem 117: 779-796
Lu B, Nagappan G, Guan X, Nathan P J, Wren P (2013) BDNF-based synaptic repair as a disease-modifying strategy for neurodegenerative diseases. Nat Rev Neurosci 14: 401-416
Marcello E, Gardoni F, Mauceri D, Romorini S, Jeromin A, Epis R, Borroni B, Cattabeni F, Sala C, Padovani A et al (2007) Synapse-associated protein-97 mediates alpha-secretase ADAM10 trafficking and promotes its activity. J Neurosci 27: 1682-1691
Meziane H, Dodart J C, Mathis C, Little S, Clemens J, Paul S M, Ungerer A (1998) Memory-enhancing effects of secreted forms of the beta-amyloid precursor protein in normal and amnestic mice. Proc Natl Acad Sci USA 95: 12683-12688
Mileusnic R, Lancashire C L, Rose S P (2004) The peptide sequence Arg-Glu-Arg, present in the amyloid precursor protein, protects against memory loss caused by A beta and acts as a cognitive enhancer. Eur J Neurosci 19: 1933-1938
Milosch N, Tanriover G, Kundu A, Rami A, Francois J C, Baumkotter F, Weyer S W, Samanta A, Jaschke A, Brod F et al (2014) Holo-APP and G-protein-mediated signaling are required for sAPPalpha-induced activation of the Akt survival pathway. Cell Death Dis 5: e1391
Mucke L, Abraham C R, Masliah E (1996) Neurotrophic and neuroprotective effects of hAPP in transgenic mice. Ann N Y Acad Sci 777: 82-88
Murakami N, Yamaki T, Iwamoto Y, Sakakibara T, Kobori N, Fushiki S, Ueda S (1998) Experimental brain injury induces expression of amyloid precursor protein, which may be related to neuronal loss in the hippocampus. J Neurotrauma 15: 993-1003
Nhan H S, Chiang K, Koo E H (2015) The multifaceted nature of amyloid precursor protein and its proteolytic fragments: friends and foes. Acta Neuropathol 129: 1-19
Obregon D, Hou H, Deng J, Giunta B, Tian J, Darlington D, Shahaduzzaman M, Zhu Y, Mori T, Mattson M P et al (2012) Soluble amyloid precursor protein-alpha modulates beta-secretase activity and amyloid-beta generation. Nat Commun 3: 777
Prokop S, Miller K R, Heppner F L (2013) Microglia actions in Alzheimer's disease. Acta Neuropathol 126: 461-477
Prox J, Rittger A, Saftig P (2012) Physiological functions of the amyloid precursor protein secretases ADAM10, BACE1, and Presenilin. Exp Brain Res 217: 331-341
Ramirez M J, Heslop K E, Francis P T, Rattray M (2001) Expression of amyloid precursor protein, tau and presenilin RNAs in rat hippocampus following deafferentation lesions. Brain Res 907: 222-232
Ring S (2007) Phenotypic analysis to define physiolocically essential functional domains of APP family proteins. In Institut für Phramazie and molekulare Biotechnologie—Funktionelle Genomik. Heidelberg: Universitat Heidelberg
Ring S, Weyer S W, Kilian S B, Waldron E, Pietrzik C U, Filippov M A, Herms J, Buchholz C, Eckman C B, Korte M et al (2007) The secreted beta-amyloid precursor protein ectodomain APPs alpha is sufficient to rescue the anatomical, behavioral, and electrophysiological abnormalities of APP-deficient mice. J Neurosci 27: 7817-7826
Roch J M, Masliah E, Roch-Levecq A C, Sundsmo M P, Otero D A, Veinbergs I, Saitoh T (1994) Increase of synaptic density and memory retention by a peptide representing the trophic domain of the amyloid beta/A4 protein precursor. Proc Natl Acad Sci USA 91: 7450-7454
Savonenko A, Xu G M, Melnikova T, Morton J L, Gonzales V, Wong M P, Price D L, Tang F, Markowska A L, Borchelt D R (2005) Episodic-like memory deficits in the APPswe/PS1dE9 mouse model of Alzheimer's disease: relationships to beta-amyloid deposition and neurotransmitter abnormalities. Neurobiol Dis 18: 602-617
Selkoe D J (2002) Alzheimer's disease is a synaptic failure. Science 298: 789-791
Selkoe, D. J. (2001). “Alzheimer's disease: genes, proteins, and therapy.” Physiol Rev 81(2): 741-766.
Shankar G M, Bloodgood B L, Townsend M, Walsh D M, Selkoe D J, Sabatini B L (2007) Natural oligomers of the Alzheimer amyloid-beta protein induce reversible synapse loss by modulating an NMDA-type glutamate receptor-dependent signaling pathway. J Neurosci 27: 2866-2875
Shankar G M, Li S, Mehta T H, Garcia-Munoz A, Shepardson N E, Smith I, Brett F M, Farrell M A, Rowan M J, Lemere C A et al (2008) Amyloid-beta protein dimers isolated directly from Alzheimer's brains impair synaptic plasticity and memory. Nat Med 14: 837-842
Spires-Jones T, Knafo S (2012) Spines, plasticity, and cognition in Alzheimer's model mice. Neural Plast 2012: 319836
Suh J, Choi S H, Romano D M, Gannon M A, Lesinski A N, Kim D Y, Tanzi R E (2013) ADAM10 missense mutations potentiate beta-amyloid accumulation by impairing prodomain chaperone function. Neuron 80: 385-401
Tang W, Ehrlich I, Wolff S B, Michalski A M, Wolfl S, Hasan M T, Luthi A, Sprengel R (2009) Faithful expression of multiple proteins via 2A-peptide self-processing: a versatile and reliable method for manipulating brain circuits. J Neurosci 29: 8621-8629
Taylor C J, Ireland D R, Ballagh I, Bourne K, Marechal N M, Turner P R, Bilkey D K, Tate W P, Abraham W C (2008) Endogenous secreted amyloid precursor protein-alpha regulates hippocampal NMDA receptor function, long-term potentiation and spatial memory. Neurobiol Dis 31: 250-260
Terry R D, Masliah E, Salmon D P, Butters N, DeTeresa R, Hill R, Hansen L A, Katzman R (1991) Physical basis of cognitive alterations in Alzheimer's disease: synapse loss is the major correlate of cognitive impairment. Annals of neurology 30: 572-580
Thornton E, Vink R, Blumbergs P C, Van Den Heuvel C (2006) Soluble amyloid precursor protein alpha reduces neuronal injury and improves functional outcome following diffuse traumatic brain injury in rats. Brain Res 1094: 38-46
Van den Heuvel C, Blumbergs P C, Finnie J W, Manavis J, Jones N R, Reilly P L, Pereira R A (1999) Upregulation of amyloid precursor protein messenger RNA in response to traumatic brain injury: an ovine head impact model. Exp Neurol 159: 441-450
Vassar R, Kuhn P H, Haass C, Kennedy M E, Rajendran L, Wong P C, Lichtenthaler S F (2014) Function, therapeutic potential and cell biology of BACE proteases: current status and future prospects. J Neurochem 130: 4-28
Villemagne V L, Burnham S, Bourgeat P, Brown B, Ellis K A, Salvado O, Szoeke C, Macaulay S L, Martins R, Maruff P et al (2013) Amyloid beta deposition, neurodegeneration, and cognitive decline in sporadic Alzheimer's disease: a prospective cohort study. Lancet Neurol 12: 357-367
Wang Y, Cella M, Mallinson K, Ulrich J D, Young K L, Robinette M L, Gilfillan S, Krishnan G M, Sudhakar S, Zinselmeyer B H et al (2015) TREM2 Lipid Sensing Sustains the Microglial Response in an Alzheimer's Disease Model. Cell 160: 1061-1071
Weyer S W, Klevanski M, Delekate A, Voikar V, Aydin D, Hick M, Filippov M, Drost N, Schaller K L, Saar M et al (2011) APP and APLP2 are essential at PNS and CNS synapses for transmission, spatial learning and LTP. EMBO J 30: 2266-2280
Weyer S W, Zagrebelsky M, Herrmann U, Hick M, Ganss L, Gobbert J, Gruber M, Altmann C, Korte M, Deller T et al (2014) Comparative analysis of single and combined APP/APLP knockouts reveals reduced spine density in APP-KO mice that is prevented by APPsalpha expression. Acta Neuropathol Commun 2: 36
Wilhelm B G, Mandad S, Truckenbrodt S, Krohnert K, Schafer C, Rammner B, Koo S J, Classen G A, Krauss M, Haucke V et al (2014) Composition of isolated synaptic boutons reveals the amounts of vesicle trafficking proteins. Science 344: 1023-1028
Wolfer D P, Stagljar-Bozicevic M, Errington M L, Lipp H P (1998) Spatial Memory and Learning in Transgenic Mice: Fact or Artifact? News Physiol Sci 13: 118-123
Xiong H, Callaghan D, Wodzinska J, Xu J, Premyslova M, Liu Q Y, Connelly J, Zhang W (2011) Biochemical and behavioral characterization of the double transgenic mouse model (APPswe/PS1dE9) of Alzheimer's disease. Neurosci Bull 27: 221-232
Yang L, Wang Z, Wang B, Justice N J, Zheng H (2009) Amyloid precursor protein regulates Cav1.2 L-type calcium channel levels and function to influence GABAergic short-term plasticity. J Neurosci 29: 15660-15668

1. A method of treating Alzheimer's disease in a subject in need thereof comprising administering to the subject a therapeutically effective amount of a vector which comprise a nucleic acid molecule encoding for a polypeptide which is a soluble member of the APP (amyloid precursor protein) family.

2. The method of claim 1 wherein the vector comprise a nucleic acid molecule that encodes for an APPsα, APLP1s or APLP2s polypeptide.

3. The method of claim 1 wherein the vector or the cell comprise a nucleic acid encoding for an APPsα polypeptide.

4. The method of claim 1 wherein the nucleic acid molecule encoding for an APPsα polypeptide comprising an amino acid sequence having at least 90% of identity with the sequence as set forth in SEQ ID NO:1 or 2.

5. The method of claim 1 wherein the nucleic acid molecule comprises a sequence having at least 70% of identity with the nucleic acid sequence as set forth in SEQ ID NO:3, or SEQ ID NO:4.

6. The method of claim 1 wherein the vector is a viral vector.

7. The method of claim 1 wherein the vector is an adeno-associated virus (AAV) vector.

8. The method of claim 7 wherein the AAV vector is selected from vectors derived from AAV serotypes having tropism for and high transduction efficiencies in cells of the mammalian central and peripheral nervous system, particularly neurons, neuronal progenitors, astrocytes, oligodendrocytes and glial cells.

9. The method of claim 7 wherein the AAV vector is an AAV4, AAV9 or an AAV10.

10. The method of claim 1 wherein the nucleic acid molecule is operatively linked to a promoter sequence.

11. The method of claim 1 wherein the vector comprises a secretory signal sequence.

12. The method of claim 1 wherein the vector comprises the nucleic acid sequence set forth in SED ID NO:5 or 6.

13. The method of claim 1 wherein the vector is delivered by intrathecal delivery.

14. A method of treating Alzheimer's disease in a subject in need thereof comprising administering to the subject a therapeutically effective amount of cells transduced with a vector which comprises a nucleic acid molecule encoding for a polypeptide which is a soluble member of the APP (amyloid precursor protein) family.

15. The method according to claim 14 wherein the cells administrated are autologous hematopoietic stem cell or hematopoietic progenitors.
